(19) United States
(12) Patent Application Publication
(10) Pub. No.: US 2003/0157094 A1
(43) Pub. Date: Aug. 21, 2003

(54) Use of il-18 inhibitors

(76) Inventors: Yolande Chvatchko, Confignon (CH); Charles Dinarello, Boulder, CO (US); Christine Plater-Zyberk, Geneva (CH); Santer Van Deventer, Haarlem (NL); Menachem Rubinstein, Givat Shmuel (IL); Daniela Novick, Rehovot (IL); Soo-Hyun Kim, Denver, CO (US)

Correspondence Address: David S Resnick, Nixon Peabody, 101 Federal Street, Boston, MA, 02110, US

(21) Appl. No.: 10/204,387

(22) PCT Filed: Feb. 20, 2001
(86) PCT No.: PCT/EP01/01867

(30) Foreign Application Priority Data
Feb. 21, 2000 (EP) 00103597.1
Feb. 21, 2000 (EP) 0103590.6
Oct. 4, 2000 (EP) 00121651.4
Nov. 23, 2000 (EP) 00125633.8

(51) Int. Cl. A61K 39/395

(52) U.S. Cl. 424/141.1; 424/145.1

(57) ABSTRACT

The invention relates to the use of inhibitors of IL-18 in the preparation of a medicament for treatment and/or prevention of liver injury. The invention further relates to the use of IL-18 inhibitors in the preparation of a medicament for treatment and/or prevention of arthritis, in particular rheumatoid arthritis. In addition to this, the invention relates to the use of inhibitors of IL-18 in the preparation of a medicament for treatment and/or prevention of inflammatory bowel diseases, in particular of Crohn's disease and ulcerative colitis.

### FIELD OF THE INVENTION

The present invention relates to the therapeutical use of IL-18 inhibitors in several pathological conditions. More specifically, the invention relates to the treatment and/or prevention of arthritis, to the treatment and/or prevention of liver diseases and to the treatment and/or prevention of inflammatory bowel diseases (IBD).

### BACKGROUND OF THE INVENTION

In 1989, an endotoxin-induced serum activity that induced interferon-γ (IFN-γ) obtained from mouse spleen cells was described (Micallef et al., 1996). This serum activity functioned not as a direct inducer of IFN-γ but rather as a co-stimulant together with IL-2 or mitogens. An attempt to purify the activity from post-endotoxin mouse serum revealed an apparently homogeneous 50-55 kDa protein. Since other cytokines can act as co-stimulants for IFN-γ production, the failure of neutralizing antibodies to IL-1, IL4, IL-5, IL-6, or TNF to neutralize the serum activity suggested it was a distinct factor. In 1995, the same scientists demonstrated that the endotoxin-induced co-stimulant for IFN-γ production was present in extracts of livers from mice preconditioned with P. acnes (Novick et al., 1992). In this model, the hepatic macrophage population (Kupffer cells) expand and in these mice, a low dose of bacterial lipopolysaccharide (LPS), which in non-preconditioned mice is not lethal, becomes lethal. The factor, named IFN-γ-inducing factor (IGIF) and later designated interleukin-18 (IL-18), was purified to homogeneity from 1,200 grams of P. acnes-treated mouse livers. Degenerate oligonucleotides derived from amino acid sequences of purified IL-18 were used to clone a murine IL-18 cDNA (Novick et al., 1992). IL-18 is an 18-19 kDa protein of 157 amino acids, which has no obvious similarities to any peptide in the databases. Messenger RNAs for IL-18 and interleukin-12 (IL-12) are readily detected in Kupffer cells and activated macrophages. Recombinant IL-18 induces IFN-gamma more potently than does IL-12, apparently through a separate pathway (Novick et al., 1992). Similar to the endotoxin-induced serum activity, IL-18 does not induce IFN-γ by itself, but functions primarily as a co-stimulant with mitogens or IL-2. IL-18 enhances T cell proliferation, apparently through an IL-2-dependent pathway, and enhances Th1 cytokine production in vitro and exhibits synergism when combined with IL-12 in terms of enhanced IFN-γ production (Maliszewski et al., 1990).

Neutralizing antibodies to mouse IL-18 were shown to prevent the lethality of low-dose LPS in P. acnes pre-conditioned mice. Others had reported the importance of IFN-γ as a mediator of LPS lethality in pre-conditioned mice. For example, neutralizing anti-IFN-γ antibodies protected mice against Shwartzman-like shock (Fantuzzi et al., 1998), and galactosamine-treated mice deficient in the IFN-γ receptor were resistant to LPS-induced death (Bym, 1990). Hence, it was not unexpected that neutralizing antibodies to murine IL-18 protected P. acnes-preconditioned mice against lethal LPS (Novick et al., 1992). Anti-murine IL-18 treatment also protected surviving mice against severe hepatic cytotoxicity.

After the murine form was cloned, the human cDNA sequence for IL-18 was reported in 1996 (Okamura et al., 1995). Recombinant human IL-18 exhibits natural IL-18 activity (Okamura et al., 1995). Human recombinant IL-18 is without direct IFN-γ-inducing activity on human T-cells, but acts as a co-stimulant for production of IFN-γ and other T-helper cell-1 (Th1) cytokines (Okamura et al., 1995). To date, IL-18 is thought of primarily as a co-stimulant for Th1 cytokine production (IFN-γ, IL-2 and granulocyte-macrophage colony stimulating factor) (Izaki, 1978) and also as a co-stimulant for FAS ligand-mediated cytotoxicity of murine natural killer cell clones (Novick et al., 1989).

By cloning IL-18 from affected tissues and studying IL-18 gene expression, a close association of this cytokine with an autoimmune disease was found. The non-obese diabetic (NOD) mouse spontaneously develops autoimmune insulitis and diabetes, which can be accelerated and synchronized by a single injection of cyclophosphamide. IL-18 mRNA was demonstrated by reverse transcriptase PCR in NOD mouse pancreas during early stages of insulitis. Levels of IL-18 mRNA increased rapidly after cyclophosphamide treatment and preceded a rise in IFN-γ mRNA, and subsequently diabetes. Interestingly, these kinetics mimic that of IL-12-p40 mRNA, resulting in a close correlation of individual mRNA levels. Cloning of the IL-18cDNA from pancreas RNA followed by sequencing revealed identity with the IL-18 sequence cloned from Kupffer cells and in vivo pre-activated macrophages. Also NOD mouse macrophages responded to cyclophosphamide with IL-18 gene expression while macrophages from Balb/c mice treated in parallel did not. Therefore, IL-18 expression is abnormally regulated in autoimmune NOD mice and closely associated with diabetes development (Novick et al., 1992).

IL-18 plays a potential role in immunoregulation or in inflammation by augmenting the functional activity of Fas ligand on Th1 cells (Conti et al., 1997). IL-18 is also expressed in the adrenal cortex and therefore might be a secreted neuro-immunomodulator, playing an important role in orchestrating the immune system following a stressful experience (Chater, 1986).

In vivo, IL-18 is formed by cleavage of pro-IL-18, and its endogenous activity appears to account for IFN-γ production in P. acnes and LPS-mediated lethality. Mature IL-18 is produced from its precursor by the IL-1β converting enzyme (IL-1beta-converting enzyme, ICE, caspase-1).

The IL-18 receptor consists of at least two components, co-operating in ligand binding. High- and low-affinity binding sites for IL-18 were found in murine IL-12 stimulated T cells (Yoshimoto et al., 1998), suggesting a multiple chain receptor complex. Two receptor subunits have been identified so far, both belonging to the IL-1 receptor family (Parnet et al., 1996). The signal transduction of IL-18 involves activation of NF-κB (DiDonato et al., 1997).

Several known cytokine binding proteins are soluble cytokine receptors and correspond to the extracellular ligand binding domains of their respective cell surface cytokine receptors. They are derived either by alternative splicing of a pre-mRNA, common to the cell surface receptor, or by proteolytic cleavage of the cell surface receptor. Such soluble receptors have been described in the past, including among others, the soluble receptors of IL-6 and IFN-γ (Nakamura et al., 1989), TNF (Dao et al., 1996; Engelmann et al., 1989), IL-1 and IL-4 (John, 1986), IFN-α/β (Mizushima and Nagata, 1990) and others. One cytokine-binding protein, named osteoprotegerin (OPG, also known as osteoclast inhibitory factor—OCIF), a member of the TNFR/Fas family, appears to be the first example of a soluble receptor that exists only as a secreted protein (Anderson, 1997; Bollon, 1980).

Recently, soluble protein having a high affinity for IL-18 has been isolated from human urine, and the human and mouse cDNAs were described (Novick et al., 1999; WO 99/09063). The protein has been designated IL-18 binding protein (IL-18BP).

IL-18BP is not the extracellular domain of one of the known IL18 receptors, but a secreted, naturally circulating protein. It belongs to a novel family of secreted proteins. The family further includes several Poxvirus-encoded proteins which have a high homology to IL-18BP (Novick et al., 1999). IL-18BP is constitutively expressed in the spleen, belongs to the immunoglobulin superfamily, and has limited homology to the IL-1 type II receptor. Its gene was localized on human chromosome 11q13, and no exon coding for a transmembrane domain was found in an 8.3 kb genomic sequence (Novick et al., 1999).

Four human and two mouse isoforms of IL-18BP, resulting from mRNA splicing and found in various cDNA libraries and have been expressed, purified, and assessed for binding and neutralization of IL-18 biological activities (Kim et al., 2000). Human IL-18BP isoform a (IL-18BPa) exhibited the greatest affinity for IL-18 with a rapid on-rate, a slow off-rate, and a dissociation constant (K(d)) of 399 pM. IL-18BPc shares the Ig domain of IL-18BPa except for the 29 C-terminal amino acids; the K(d) of IL-18BPc is 10-fold less (2.94 nM). Nevertheless, IL-18BPa and IL-18BPc neutralize IL-18 >95% at a molar excess of two. IL-18BPb and IL-18BPd isoforms lack a complete Ig domain and lack the ability to bind or neutralize IL-18. Murine IL-18BPc and IL-18BPd isoforms, possessing the identical Ig domain, also neutralize >95% murine IL-18 at a molar excess of two. However, murine IL-18BPd, which shares a common C-terminal motif with human IL-18BPa, also neutralizes human IL-18. Molecular modelling identified a large mixed electrostatic and hydrophobic binding site in the Ig domain of IL-18BP, which could account for its high affinity binding to the ligand (Kim et al., 2000).

Recently, it has been suggested that the interleukin IL-18 is involved in the progression of pathogenicity in chronic inflammatory diseases, including endotoxin shock, hepatitis, and autoimmune-diabetes (Kahiwamura and Okamura, 1998). A further indication of a possible role of IL-18 in the development of liver injury resulted from experiments published by Tsuij et al. (Tsuij et al., 1999), showing an elevated tevel of IL-18 in lipopolysaccharide-induced acute liver injury in a mouse model. However, the mechanism of the multi-functional factor IL-18 in the development of liver injury has not been elucidated so far.

Liver damage or injury may have diverse causes. It may be due to viral or bacterial infections, alcohol abuse, immunological disorders, or cancer, for example.

Viral hepatitis, due to Hepatitis B virus and Hepatitis C virus, for example, are poorly managed diseases that afflict large number of people world-wide. The number of known hepatitis viruses known is constantly increasing. Apart from Hepatitis B and C virus, at least four other viruses causing virus-associated hepatitis have been discovered so far, called Hepatitis A, D, E and G-Virus.

Alcoholic liver disease is another widespread disease associated with chronic consumption of alcohol. Immune hepatitis is a rare autoimmune disease that is poorly managed. Liver injury also includes damages of the bile ducts. Primary biliary cirrhosis (PBC) is an autoimmune liver disease characterized by destruction of the intrahepatic bile ducts.

Several studies have demonstrated that damage to the liver in diseases such as alcoholic hepatitis, liver cirrhosis, viral hepatitis and primary biliary cirrhosis is associated with T-helper cell-1 (Th1) responses. In one study, a novel liver injury model was established in mice by targeting of ovalbumin-containing liposomes into the liver, followed by adoptive transfer of ovalbumin-specific Th1 cells. Combined treatment of mice with ovalbumin-containing liposomes and Th1 cell transfer caused an increase in serum transaminase activity that was paralleled with an elevation of serum IFN-γ levels. In sharp contrast, ovalbumin-specific Th2 cell transfer resulted in an increase of serum IL-4 levels but did not induce liver injury. The liver injury was blocked by anti-IFN-γ antibodies and anti-tumor necrosis factor (TNF)-α antibodies. These findings indicate that Th1 cells are the major effector cells in acute liver injury (Nishimura and Ohta, 1999) In another set of studies it was shown that mice over-expressing IFN-γ exhibit spontaneous hepatitis without any pathogen or any other stimulant (Okamoto et al., 1998).

Another study implicated Th1 responses in primary biliary cirrhosis (PBC). PBC is an autoimmune liver disease characterized by destruction of the intrahepatic bile ducts. It is generally believed that cellular immune mechanisms, particularly involving T cells, result in this bile duct damage. The relative strength of Th1 and Th2 responses has recently been proposed to be an important factor in the pathophysiology of various autoimmune diseases. In this study, the subset balance in PBC was evaluated by detection of cytokines specific to the two T-cell subsets, i.e., IFN-γ for Th1 cells and IL-4 for Th2 cells. IFN-γ and IL-4 messenger RNA (mRNA) positive cells were counted in liver sections from 18 patients with PBC and 35 disease controls including chronic active hepatitis C, extrahepatic biliary obstruction, and normal liver, using nonisotopic in situ hybridization and immunohistochemistry. Mononuclear cells expressing IFN-γ and IL-4 mRNA were aggregated in inflamed portal tracts in PBC livers, but were rarely present in extrahepatic biliary obstruction, alcoholic fibrosis, or normal liver sections. The IFN-γ and IL-4 mRNA positive cells in PBC livers were detected in significantly higher numbers than in control livers (P<0.01). Moreover, IFN-γ mRNA expression was more commonly detected than IL-4 expression in PBC livers, and the levels of IFN-γ mRNA expression were highly correlated with the degree of portal inflammatory activity. IFN-γ mRNA-positive cells were detected primarily around damaged bile ducts that were surrounded by lymphoid aggregates. The data indicate that Th1 cells are the more prominent T-cell subset in the lymphoid infiltrates in PBC (Harada et al., 1997).

The cytokine pattern on viral antigen recognition is also believed to exert a profound influence on the resolution of viral infections and viral clearance. One study investigated whether a cytokine imbalance oriented toward Th2 type response plays a role in chronic hepatitis B. Cytokine profiles of peripheral blood mononuclear cells associated with chronic hepatitis B were analyzed by RT-PCR. Upon hepatitis B surface antigen (HbsAg) stimulation, expression of IFN-γ, IL-2, IL-4, and IL-10 was detected in 41%, 8%, 41%, and 50% of the patients, respectively. Among these cytokines, the expression of the Th1 cytokine IFN-γ was associated with high levels of serum AST/ALT (Aspartate aminotransferase/Alanine aminotransferase), representing typical markers of liver damage. Th2 type cytokines were not shown to exert a protective effect on hepatocytes. In conclusion, production of a Th1 cytokine, IFN-γ, by HBsAg-reactive cells was associated with hepatocyte damage in chronic hepatitis B (Lee et al., 1999). High levels of the FAS ligand and its receptor (CD95) were reported in liver of hepatitis B patients (Luo et al., 1997). FAS ligand is considered to be one of the major cytotoxic agents leading to hepatocyte apoptosis.

Another study identified factors associated with the progression of liver injury in 30 hepatitis C virus/RNA (HCV/RNA)-positive untreated patients with chronic hepatitis. Necroinflammatory and architectural damage were evaluated using Ishak's score. Activated hepatic stellate cells (HSC) were visualized by immunohistochemistry for α-smooth muscle actin (αSMA) and quantitated by morphometry. Plasma HCV/RNA was evaluated using a competitive RT-PCR method. To study the type of immune response involved in the progression of liver injury, IFN-γ-positive cells (as expression of a Th1-like response) were evaluated by immunohistochemistry and quantitated by morphometry. It was found that HSC were mostly detected close to areas of lobular necroinflammation or lining fibrotic septa. The αSMA- and Sirius Red-positive parenchyma correlated significantly with necroinflammatory and architectural scores. IFNγ-positive cells were detected in periportal areas associated with the inflammatory infiltrates and significantly correlated with architectural damage. It was therefore concluded that HSC activation and progression of liver injury are associated with a Th1-like response (Baroni et al, 1999). Similarly to the case of Hepatitis B, FAS ligand and its receptor were found in liver and sera of hepatitis C patients (Hiramatsu et al, 1994; Okazaki et al, 1996; Lio et al., 1998)

Th1 cytokines and other Th1 markers were found to be associated with alcoholic hepatitis and liver cirrhosis. Inflammatory stimuli and lipid peroxidation activate nuclear factor κ B (NF-κB) and upregulate proinflammatory cytokines and chemokines. In one study, the relationship between pathological liver injury, endotoxemia, lipid peroxidation, and NF-κB activation and imbalance between pro- and anti-inflammatory cytokines was evaluated. Rats (5 per group) were fed ethanol and a diet containing saturated fat, palm oil, corn oil, or fish oil by intragastric infusion. Dextrose isocalorically replaced ethanol in control rats. Pathological analysis was performed and measurements of endotoxin were taken, lipid peroxidation, NF-κB, and messenger RNA (mRNA) levels of proinflammatory cytokines (TNFα, IL-1beta, IFN-γ, and IL-12), C-C chemokines (regulated upon activation, normal T cell expressed and secreted [RANTES], monocyte chemotactic protein [MCP]-1, macrophage inflammatory protein [MIP]-1-α), C-X-C chemokines (cytokine induced neutrophil chemoattractant [CINC], MIP-2, IP-10, and epithelial neutrophil activating protein [ENA]-78), and anti-inflammatory cytokines (IL-10, IL-4, and IL-13). Activation of NF-κB and increased expression of proinflammatory cytokines C-C and C-X-C chemokines was seen in the rats exhibiting necroinflammatory injury (fish oil-ethanol and corn oil-ethanol). These groups also had the highest levels of endotoxin and lipid peroxidation. Levels of IL-10 and IL-4 mRNA were lower in the group exhibiting inflammatory liver injury. Thus, activation of NF-κB occurs in the presence of proinflammatory stimuli and results in increased expression of Th1 proinflammatory cytokines and chemokines (Naji et al., 1999). FAS ligand and its receptor are also elevated in alcoholic liver diseases, suggesting once again that Th1 cytokines are involved in the autoimmune processes induced in alcoholic hepatitis (Galle et al., 1995; Taieb et al, 1998; Fiore et al., 1999).

TNF-α has also emerged as a common pathway in the pathogenesis of alcohol-related hepatic necro-inflammation. Increased levels of hepatic and serum TNF have been documented in animal models of alcoholic liver disease and in human alcoholic liver disease. This dysregulated TNF metabolism has been postulated to play a role in many of the metabolic complications and the liver injury of alcoholic liver disease (Grove et al., 1997; McClain and Cohen, 1989). For instance it was found in one study that patients with alcoholic hepatitis had higher TNF-α levels (mean, 26.3 ng/L; 95% Cl, 21.7 to 30.9) than normal subjects (6.4 ng/L; Cl, 5.4 to 7.4). Patients who subsequently died had a higher TNF-α level (34.7 ng/L; Cl, 27.8 to 41.6) than survivors (16.6 ng/L; Cl, 14.0 to 19.2). In patients with alcoholic hepatitis, TNF-α levels correlated positively with serum bilirubin (r=0.74; P=0.0009) and serum creatinine (r=0.81; P=0.0003). Patients with alcoholic hepatitis had higher TNF-α levels than patients with inactive alcoholic cirrhosis (11.1 ng/L; Cl, 8.9 to 13.3) and severely alcoholic persons without liver disease (6.4 ng/L; Cl, 5.0 to 7.8). Patients with abnormal renal function had lower TNF-α levels (14.1 ng/L; Cl, 5.4 to 22.8) than patients with alcoholic hepatitis. It was therefore concluded that elevations in TNF-α in alcoholic hepatitis are most marked in severe cases, suggesting that TNF-α plays a role in the pathogenesis (Bird et al., 1990)

TNF mediates many of the biologic actions of endotoxin. Recent studies have shown that TNF administration may cause liver injury and that TNF may mediate the lethality of the hepatotoxin galactosamine. One of the most potent TNF inducers is endotoxin. Because patients with alcoholic liver disease frequently have endotoxemia and because many of the clinical manifestations of alcoholic hepatitis are known biologic actions of TNF, its activity was evaluated in patients with alcoholic hepatitis. Basal and lipopolysaccharide-stimulated TNF release from peripheral blood monocytes, a major source of TNF production, was determined in 16 patients with alcoholic hepatitis and 16 healthy volunteers. Eight of 16 alcoholic hepatitis patients and only two of 16 healthy volunteers had detectable spontaneous TNF activity (p less than 0.05). After lipopolysaccharide stimulation, mean monocyte TNF release from alcoholic hepatitis patients was significantly increased to over twice that of healthy controls (25.3±3.7 vs. 10.9±2.4 units per ml, p less than 0.005). It was therefore concluded that monocytes from alcoholic hepatitis patients have significantly increased spontaneous and lipopolysaccharide-stimulated TNF release compared to monocytes from healthy volunteers (McClain and Cohen, 1989.

Lipopolysaccharide (LPS)-binding protein (LBP) and CD14 play key intermediary roles in the activation of cells by endotoxin. Gut-derived LPS has been postulated to participate in promoting pathological liver injury in alcoholic liver disease. It was demonstrated that rats fed intragastrically with ethanol in oil for 4 weeks had elevated levels of CD14 and LBP in their Kupffer cells and hepatocytes, respectively. Expression of CD14 mRNA was also elevated in nonmyeloid cells. Enhanced LBP and CD14 expression rapidly increases the LPS-induced expression of various pro-inflammatory cytokines and correlates with the presence of pathological liver injury in alcoholic liver injury (Su et al., 1998; Lukkari et al., 1999).

Arthritis is a disease involving joint inflammation. The joints show swelling, stiffness, tenderness, redness or warmth. The symptoms may be accompanied by weight loss, fever or weakness. When these symptoms last for more than two weeks, inflammatory arthritis e.g. rheumatoid arthritis may be the cause. Joint inflammation may also be caused by infection, which can lead to septic arthritis. A very common type of arthritis is degenerative joint disease (osteoarthritis).

The medicaments commonly prescribed for arthritis and related conditions are non-steroidal anti-inflammatory drugs (NSAIDs). NSAIDs include aspirin and aspirin-like drugs. They reduce inflammation, which is the cause for joint pain, stiffness and swelling of the joints. However, NSAIDs are unspecific drugs having a number of side effects, involving bleeding of the stomach (Homepage of the Department of Orthopaedics of the University of Washington on Arthritis, Frederick Matsen (Chairman), www.orthop.washington.edu). In addition to NSAIDs, Celebrex™, a cyclooxygenase (COX-2) inhibitor, is used to relieve the signs and symptoms of osteoarthritis and rheumatoid arthritis in adults. It is also indicated for the treatment of patients with familial adenomatous polyposis.

WO 01/00229 describes a combination of tumors necrosis factor (TNF) antagonists and COX-2 inhibitors for the treatment of inflammation.

TNF antagonists are alsos used for the treatment of arthritis. TNF antagonists are described, for example, in WO 9103553.

Recent studies indicate that the interleukin IL-18 plays a proinflammatory role in joint metabolism. Olee et al. (1999) showed that IL-18 is produced by articular chondrocytes and induces proinflammatory and catabolic responses. The IL-18 mRNA was induced by IL-1β in chondrocytes. Chondrocytes produced the IL-18 precursor and in response to IL-1 stimulation secreted the mature form of IL-18. Studies on IL-18 effects on chondrocytes further showed that it inhibits TGF-β-induced proliferation and enhances nitric oxide production. IL-18 stimulated the expression of several genes in normal human articular chondrocytes including inducible nitric oxide synthase, inducible cyclooxygenase, IL-6, and stromelysin. Gene expression was associated with the synthesis of the corresponding proteins. Treatment of normal human articular cartilage with IL-18 increased the release of glycosaminoglycans. These finding identified IL-18 as a cytokine that regulates chondrocyte responses and contributes to cartilage degradation.

The localisation of Interleukin-1β-converting enzyme (ICE)/caspase-1 in human osteoarthritic tissues and its role in the maturation of interleukin-1beta and interleukin-18 have been shown by Saha et al. (1999). Saha et al. studied the expression and production of caspase-1 in human normal and osteoarthritic (OA) cartilage and synovium, quantitated the level of ICE in OA chondrocytes, and examined the relationship between the topographic distribution of ICE, interleukin-1β (IL-1β), and IL-18, as well as apoptosis of chondrocytes. The experiments performed in this study indicated that ICE was expressed and synthesised in both human synovial membrane and cartilage, with a significantly greater number of cells staining positive in OA tissue than in normal tissue. ICE production was preferentially located in the superficial and upper intermediate layers of articular cartilage. The production of mature IL-1beta in OA cartilage explants and chondrocytes was completely blocked by treatment with a specific ICE inhibitor, which also markedly diminished the number of IL-18-positive cells. The relationship between active IL-1beta and ICE suggests that ICE may promote OA progression by activating this proinflammatory cytokine, and that IL-18 may play a role in cartilage pathology.

Gracie et al. (1999) suggested a proinflammatory role for IL-18 in rheumatoid arthritis. Gracie et al. detected the IL-18 mRNA and protein within rheumatoid arthritis synovial tissues in significantly higher levels than in osteoarthritis controls. It was also shown that a combination of IL-12 or IL-15 with IL-18 induced the IFN-γ production by synovial tissues in vitro. Furthermore, IL-18 administration of collagen/inclomplete Freund's adjuvant-immunized mice facilitated the development of an erosive, inflammatory arthritis, suggesting that IL-18 may be proinflammatory in vivo.

However, so far, apart from chemical compounds, only the blockade of TNFα and IL-1β by using soluble receptors or monoclonal antibodies have been shown to decrease murine collagen-induced arthritis (CIA, which is a mouse model for rheumatoid arthritis) (Williams et al., 1994), and were therefore suggested as a therapeutic for rheumatoid arthritis.

The term “chronic or idiopathic inflammatory bowel diseases” embraces at least two conditions: Crohn's disease and ulcerative colitis. Both are diseases of the gastrointestinal tract, Crohn's disease most commonly affecting the small bowel. When it also involves the colon, the differential diagnosis from ulcerative colitis (see below) can be a problem.

The chronic inflammation and ulceration in Crohn's disease usually starts with either small-intestinal obstruction or abdominal pain which may mimic acute appendicitis; other presentations can relate to its complications. The course of the disease is chronic, and there may be exacerbations and remissions in spite of therapy. Onset is usually in early adult life, with about half of all cases beginning between the ages of 20 and 30 years and 90% between 10 and 40 years. Slightly more males than females are affected.

Microscopy reflects the gross appearances. Inflammation involvement is discontinuous: it is focal or patchy. Collections of lymphocytes and plasma cells are found mainly in the mucosa and submucosa but usually affecting all layers (transmural inflammation). The classical microscopic feature of Crohn's disease is the presence of granule cells surrounded by a cuff of lymphocytes. The incidence of idiopathic inflammatory bowel diseases shows considerable geographic variation. These diseases have a much higher incidence in northern Europe and the United States than in countries of southern Europe, Africa, South America and Asia, although increasing urbanisation and prosperity is leading to a higher incidence in parts of southern Europe and Japan (General and Systematic Pathology, Churchill Livingstone, 3^{rd} edition 2000, J C E Underwood, Ed.).

In Crohn's disease, clinically there are two main groups, the first comprising patients whose disease goes into lasting remission within three years of onset, the second comprising patients with disease persisting beyond three years.

Whatever the aetiology, there is evidence of persistence and inappropriate T-cell and macrophage activation in Crohn's disease with increased production of proinflammatory cytokines, in particular interleukins (IL) 1, 2, 6 and 8, Interferon (IFN)-γ and Tumor Necrosis Factor (TNF) α. Crohn's disease is characterised by sustained (chronic) inflammaton accompanied by fibrosis. The process of fibroblastic proliferation and collagen deposition may be mediated by transforming growth factors, which has certain anti-inflammatory actions, namely fibroblast recruitment, matrix synthesis and down-regulation of inflammatory cells, but it is likely that many other mediators will be implicated.

Ulcerative colitis is a non-specific inflammatory disorder of the large intestine, usually beginning in the rectum and extending proximately to a varying extent. Unlike Crohn's disease, ulcerative colitis is confined to the large intestine.

There is growing evidence to indicate that ulcerative colitis is a consequence of altered autoimmune reactivity but mucosal injury could also result from inappropriate T-cell activation and indirect damage brought about by cytokines, proteases and reactive oxygen metabolites from macrophages and neutrophils. This latter mechanism of damage to the colonic epithelium has been termed “innocent bystander” injury. Evidence in favour of autoimmunity is the presence of self-reactive T-lymphocytes and auto-antibodies directed against colonic epithelial cells and endothelial cells, and anti-neutrophil cytoplasmic auto-antibodies (ANCA). However, ulcerative colitis should not be thought of as an autoimmune disease in which mucosal injury is a direct consequence of an immunological reaction to self-antigens (General and Systematic Pathology, supra).

With regard to the therapy of Crohn's disease, most people are first treated with drugs containing mesalamine, a substance that helps control inflammation. Patients who do not benefit from it or who cannot tolerate it may be put on other mesalamine-containing drugs, generally known as 5-ASA agents. Possible side effects of mesalamine preparations include nausea, vomiting, heartburn, diarrhea, and headache.

Some patients take corticosteroids to control inflammation. These drugs are the most effective for active Crohn's disease, but they can cause serious side effects, including greater susceptibility to infection.

Drugs that suppress the immune system are also used to treat Crohn's disease. Most commonly prescribed are 6-mercaptopurine and a related drug, azathioprine. Immunosuppressive agents work by blocking the immune reaction that contributes to inflammation. These drugs may cause side effects like nausea, vomiting, and diarrhea and may lower a person's resistance to infection. When patients are treated with a combination of corticosteroids and immunosuppressive drugs, the dose of corticosteriods can eventually be lowered. Some studies suggest that immunosuppressive drugs may enhance the effectiveness of corticosteroids.

The U.S. Food and Drug Administration has approved the drug infliximab for the treatment of moderate to severe Crohn's disease that does not respond to standard therapies (mesalamine substances, corticosteroids, immunosuppressive agents) and for the treatment of open, draining fistulas. Infliximab, the first treatment approved specifically for Crohn's disease, is an anti-tumor necrosis factor (TNF) monoclonal antibody. Anti-TNF removes TNF from the bloodstream before it reaches the intestines, thereby preventing inflammation.

Antibiotics are used to treat bacterial overgrowth in the small intestine caused by stricture, fistulas, or prior surgery. For this common problem, the doctor may prescribe one or more of the following antibiotics: ampicillin, sulfonamide, cephalosporin, tetracycline, or metronidazole.

Diarrhea and crampy abdominal pain are often relieved when the inflammation subsides, but additional medication may also be necessary. Several anti-diarrhea agents could be used, including diphenoxylate, loperamide, and codeine. Patents who are dehydrated because of diarrhea are usually treated with fluids and electrolytes.

There remains to be a need for effective therapy for the treatment and/or prevention of inflammatory bowel diseases, in particular Crohn's disease and ulcerative colitis, which have reduced side effects or are ideally even free of side effects.

Both histological and immunological observations indicate that cell-mediated immunity and T cell activation are key features of CD. Studies from humans and experimental models suggest that, in CD, the local immune response tends to be predominantly Th1 in type (Desreumaux, et al. 1997) and that locally released cytokines, such as IFN-γ, IL-1β, and TNF-α, contribute to promote and expand the inflammatory response (Reimund et al. 1996).

The cytokine IL-18 plays an important role in Th1 mediated immune response in collaboration with the cytokine IL-12 by stimulating IFN-γ secretion, enhancing natural killer cell cytotoxicity, and stimulating TH1 cell differentiation (Uschito et al, 1996).

IL-18 acts together with IL-12, IL-2, antigens, mitogens, and possibly further factors, to induce the production of IFN-γ. IL-18 also enhances the production of GM-CSF and IL-2, potentiates anti-CD3 induced T cell proliferation, and increases Fas-mediated killing of natural killer cells. Mature IL-18 is produced from its precursor by the IL-1β converting enzyme (ICE, caspase-1). The IL-18 receptor consists of at least two components, co-operating in ligand binding. High- and low-affinity binding sites for IL-18 were found in murine IL-12 stimulated T cells (Okamoto et al., 1998), suggesting a multiple chain receptor complex. Two receptor subunits have been identified so far, both belonging to the IL-1 receptor family (Okamoto et al., 1999). The signal transduction of IL-18 involves activation of NF-κB {Matsumoto, et al. 1997).

Recently, IL-18 has been suggested to have some implication in Inflammatory Bowel Diseases (Pizarro, et al. 1999; Monteleone, et al. 1999).

Pizarro et al. (1999) characterised the expression and localisation of IL-18 in colonic specimens and isolated mucosal cell populations from patients with Crohn's disease. Using a semiquantitative RT-PCR protocol, IL-18 mRNA transcripts were found to be increased in freshly isolated intestinal epithelial cells and lamina propria mononuclear cells from CD compared with ulcerative colitis and noninflamed control patients. IL-18 mRNA transcripts were more abundant in intestinal epithelial cells compared with lamina propria mononuclear cells. Immunohistochemical analysis of surgically resected colonic tissues localised IL-18 to both lamina propria mononuclear cells (specifically, macrophages and dendritic cells) as well as intestinal epithelial cells. Western blot analysis revealed that an 18.3-kDa band, consistent with both recombinant and mature human IL-18 protein, was found predominantly in CD vs UC intestinal mucosal biopsies; a second band of 24 kDa, consistent with the inactive IL-18 precursor, was detected in noninflamed areas from both CD and UC biopsies and was the sole form found in noninflamed controls.

Monteleone et al. (1999) confirmed these findings. Whole mucosal intestinal tissue and lamina propria mononuclear cells of 12 Crohn's disease and 9 ulcerative colitis patients and 15 non-inflammatory bowel disease controls were tested for IL-18 by semiquantitative RT-PCR and Western blot analysis. Transcripts for IL-18 were found in all samples tested. However, increased IL-18 mRNA accumulation was detected in both mucosal and lamina propria mononuclear cells samples from Crohn's disease in comparison to ulcerative colitis and controls. In Crohn's disease, transcripts for IL-18 were more abundant in the mucosal samples taken from involved areas. An 18-kDa band consistent with mature IL-18 was predominantly found in Crohn's disease mucosal samples. In mucosal samples from non-IBD controls, IL-18 was present as the 24-kDa polypeptide. Consistently, active IL-1beta-converting enzyme (ICE) subunit (p20) was expressed in samples from either CD or UC, whereas, in colonic mucosa from non-IBD controls, ICE was synthesised as precursor (p45) only.

Dayer (1999) reviewed the different and partially contradicting functions of IL-18. In summary, IL-18 is a pleiotropic interleukin having both inflammatory enhancing and attenuating functions. On the one hand, it enhances production of the proinflammatory cytokines like TNFα, therefore promoting inflammation. On the other hand, it induces the production of NO, an inhibitor of caspase-1, thus blocking the maturation of IL-1β and IL-18, and possibly attenuating inflammation. This ambiguous role of IL-18 seriously questioned the efficacy of IL-18 inhibitors in inflammatory diseases. Furthermore, because of the interaction of a huge variety of different cytokines and chemokines in the regulation of inflammation, a beneficial effect in therapy or prevention of inflammatory diseases by blocking only one of the players is not predictable.

### SUMMARY OF THE INVENTION

The present invention is based on the finding that inhibitors of IL-18 are effective for treatment and/or prevention of different diseases or disorders.

It is a first object of the present invention to provide for a novel means for treating and/or preventing liver injury. The invention therefore relates to the use of an IL-18 inhibitor for the manufacture of a medicament for treatment and/or prevention of liver injury, be it acute or chronic. More specifically, the invention relates to the treatment and/or prevention of alcoholic hepatitis, viral hepatitis, immune hepatitis, fulminant hepatitis, liver cirrhosis, and primary biliary cirrhosis.

It is a second object of the present invention to provide for a novel means for treating and/or preventing arthritis. The invention therefore also relates to the use of IL-18 inhibitors in the preparation of a medicament for treatment and/or prevention of arthritis. The beneficial effect of IL-18 inhibitors includes decreasing the severity of the disease, as well as preventing the spreading of the disease. This finding is unexpected, since from the state of the art outlined above, it could not have been concluded that a blockade of one specific factor involved in arthritis, namely interleukin IL-18, would lead to the alleviation of arthritis or even the curing of a diseased arthritic joint.

It has also been found that the administration of an IL-18 inhibitor significantly diminishes cartilage erosion in a murine model of arthritis. The present invention thus further relates to the use of an inhibitor of IL-18 in the manufacture of a medicament for treatment and/or prevention of cartilage destruction.

It is a third object of the present invention to provide for a novel means for treating and/or preventing inflammatory bowel disease (IBD), in particular Crohn's disease and ulcerative colitis. The invention therefore also relates to the use of an IL-18 inhibitor for the manufacture of a medicament for treatment and/or prevention of IBD. In accordance with the present invention it has now been found that the concentrations of IL-18BP mRNA and protein are increased in inflamed regions of the mucosa in biopsies derived from Crohn's disease patients. Further, it has been shown that two different inhibitors of IL-18 protected animals from disease in a murine model of inflammatory bowel disease.

The use of combinations of an IL-18 inhibitor and/or an interferon and/or a TNF antagonist and/or a COX-2 inhibitor are also considered according to the invention. In order to apply gene therapeutical approaches to deliver the IL-18 inhibitor to diseased tissues or cells, further aspects of the invention relate to the use of expression vectors comprising the coding sequence of an IL-18 inhibitor for the treatment and/or prevention of the disease conditions. The invention further relates to the use of endogenous gene activation of IL-18 inhibitors and to the use of cells genetically engineered to express IL-18 inhibitors for the prevention and/or treatment of liver injury, arthritis and IBD.

### BRIEF DESCRIPTION OF THE DRAWINGS

FIG. 1 shows a histogram depicting the serum levels of IFN-γ (pg/ml) after injection of various amounts of recombinant IL18BP (0; 0.04; 0.4; 4 mg/kg) into mice 1 h before the injection of LPS. Blood samples were taken 5 h after LPS injection and analyzed by ELISA for circulating IFN-γ.

FIG. 2 shows a histogram depicting the serum levels of Alanine aminotransferase (ALT). Mice were injected with increasing doses of recombinant human IL18BP (0; 0.04; 0.4; 4 mg/kg) before injection of LPS into P. acnes sensitized mice. Blood samples were taken 5 h after LPS injection and serum levels of ALT were measured. SF=Sigma-Frankel: 1 SF Unit of AST/ALT will form 4.82×10⁻⁴ μmol glutamate/minute at pH 7.5 at 25° C.

FIG. 3 shows the survival time of the mice after LPS injection. Mice were injected with different doses of recombinant human IL18BP (0; 0.04; 0.004; 4 mg/kg) 20 min before injection of LPS into P. acnes sensitized mice. Triangles: 4 mg/kg; small diamond: 0.4; big diamond: 0.04; circles: no IL18BP (only LPS).

FIG. 4 shows a histogram depicting serum levels of IFN-γ, measured 5 h after injection of different amounts of IL18BP (0; 0.4; 4 mg/kg), which was administered 20 min before LPS injection into P. acnes sensitized mice.

FIG. 5 shows the survival of mice injected either with polyclonal IL-18 antiserum or normal rabbit serum (NDS=control) 30 min before injection with 40 mg/ml (lethal dosis) of LPS derived from E. coli (FIG. 5 A) or S. thyphimurium (FIG. 5 B). Triangles: mice were injected with IL-18 antiserum; circles: mice were injected with NDS. On the x-axis, the days after LPS challenge are depicted. * p<0.05.

FIG. 6 shows a histogram, depicting the mean+SEM of five mice per group treated in the following way. Mice were injected intraperitoneally (i.p.) either anti-IL-18 antiserum, soluble TNF-α receptors (TNFsRp55) or vehicle (saline), immediately followed by the intravenous (i.v.) administration of Concanavalin A (Con A; FIG. 6 A) or PEA (Pseudomonas aeruginosa, FIG. 6 B). **p<0.01; ***p<0.001 vs ConA or PEA alone; # p<0.01 vs either TNFsRp55 or anti-IL-18 factorial ANOVA.

FIG. 7 shows the effect of of IL-18BP on clinical scores in a murine model of arthritis. FIG. 7 A shows a diagram depicting the clinical scores measured after daily administration of different amounts of IL-18BP or IFN-β or vehicle (NaCl) i.p. (intraperitoneally) to mice. Symbols: Filled triangles: 10000 IU IFN-β; open triangles: 10 mg/kg IL-18BP, reversed triangles: 3 mg/kg IL-18BP, diamonds: 1 mg/kg IL-18BP; circles: 0.5 mg/kg IL-18BP; open squares: 0.25 mg/kg IL-18BP, and filled squares: NaCl. The days of treatment are depicted on the x-axis, the clinical scores (mean values) are depicted on the y-axis. Statistics were calculated by the Mann Whitney test. FIG. 7 B shows a histogram depicting the AUC (area under the curve) derived from the graph of FIG. 7 A. n=number of animals.

FIG. 8 shows the effect of IL-18BP on paw swelling. FIG. 8 A shows a diagram depicting the results obtained by measuring the paw thickness (swelling) of diseased hind paws of individual animals treated with different amounts of IL-18BP. The y-axis shows the change of paw thickness in millimeters from the beginning of treatment. The symbols are as in FIG. 7. FIG. 8 B shows a histogram depicting the AUC derived from FIG. 8 A. n=number of animals.

FIG. 9 shows the analysis of the number of diseased hind paws at the time of acute arthritis, i.e. spreading of the disease to additional joints. Symbols: Filled squares: NaCl (control), triangles: 10 mg/kg IL-18BP, reversed triangles: 3 mg/kq IL-18BP, diamonds: 1 mg/kg IL-18BP, circles: 0.5 mg/kg IL-18BP and open squares: 0.25 mg/kg IL-18BP.

FIG. 10 shows a histogram depicting the erosion scores of the cartilage of diseased joints.

FIG. 11 shows the histopathology of mouse joints. At the end of the experiment, the paw that first developed arthritis was dissected away, fixed and processed as described in Example 10 below. FIG. 11 A: normal mouse joint; FIG. 11 B: joint from an arthritic mouse; FIG. 11 C: joint from a mouse treated with rhIL-18BP.

FIG. 12 shows a histogram depicting the levels of anti-collagen type II antibodies of the isotype IgG1 (open columns) or IgG2a (hatched columns) of mice treated with 3 mg/kg of IL-18BP or saline (vehicle), respectively. Measurements were taken on day 4 (D4) or day 8 (D8) of the disease.

FIG. 13 shows a histogram depicting IL-6 levels in pg/ml of animals treated with 1, 3 or 10 mg/kg of IL-18BP, 10 000 IU of Interferon β (IFN-b), normal mouse serum (NMS) or saline (NaCl), respectively.

FIG. 14 shows the expression of hIL-18BP and IL-18 mRNA transcripts in intestinal biopsies from patients suffering from active Crohn's disease, ulcerative colitis or normal healthy individuals. Representative RT-PCR products are shown for IL-18BP, for IL-18 and for a housekeeping gene (β-actin) (FIG. 14 A). Relative quantification of ethidiumbromide-stained bands was carried out using the Kodak Digital Imaging Software and are reported as the ratio of target gene to β-actin. The target gene is IL-18 in FIG. 14 B and IL-18BP in FIG. 14 C.

FIG. 15 shows the expression of hIL-18BP mRNA transcripts and of protein by HUVECs (human umbilical vein endothelial cells) and the expression of protein by THP1 (human monocytic cell line). RNA was isolated from non-treated endothelial cells (medium) and endothelial cells stimulated with IL-1β, TNFα, IFNγ. Positive control: colon from patient with Crohn's disease, negative control: without cDNA. IL-18BP and IL-18 expression was analysed by semiquantitative RT-PCR (FIG. 15 A). Culture supernatant from non-treated (medium) and upon 24 h activation with IL-1β, TNFα, IFNγ of HUVEC (FIG. 15 B) or THP1 (FIG. 15 C) cells were analysed for IL-18BP and IL-18 protein production by ELISA.

FIG. 16 shows the development of bodyweight between day 1 and day 10 in a mouse model of IBD after intraperitoneal (ip) administration of either saline (NaCl) or IL-18BP (8 mg/kg). The change in weight is expressed as percentage of the body weight change from day 1. Mean values and SEM of two groups are shown, 8 mice per group.

FIG. 17 shows the results of analyses of colons, caudal lymph nodes and spleen derived from IL-18BP treated vs. non-treated IBD mice. FIG. 17 A depicts the weight of the last 6 centimetres of colon in mg. FIG. 17 B shows the total number of cells present in the caudal lymph node. FIG. 17 C depicts the percentage of cells staining positive for CD4⁺/CD69⁺ in the spleen. Data represent mean values and SEM. * indicates a significant difference.

FIG. 18 shows the amount of IFNγ (FIGS. 18 A and B) and TNFα (FIGS. 18 C and D) produced after 48 hours by caudal lymph node cells (FIGS. 185 A and C) and spleen cells (FIGS. 18 B and D) after stimulation with CD3/CD28 present in the supernatants. Mean and SEM are shown.

FIG. 19 shows the TNFα (FIG. 19 A) and IFNγ (FIG. 19 B) content in colon homogenates. Data are corrected for the colon weight. Mean values and SEM are shown. * indicates a significant difference.

### DESCRIPTION OF THE INVENTION

The present invention is based on the finding of a beneficial effect of an inhibitor of IL-18 in different diseases and disorders.

The term “inhibitor of IL-18” within the context of this invention refers to any molecule modulating IL-18 production and/or action in such a way that IL-18 production and/or action is attenuated, reduced, or partially, substantially or completely prevented or blocked. The term “IL-18 inhibitor” is meant to encompass inhibitors of IL-18 production as well as of inhibitors of IL-18 action.

An inhibitor of production can be any molecule negatively affecting the synthesis, processing or maturation of IL-18. The inhibitors considered according to the invention can be, for example, suppressors of gene expression of the interleukin IL-18, antisense mRNAs reducing or preventing the transcription of the IL-18 mRNA or leading to degradation of the mRNA, proteins impairing correct folding, or partially or substantially preventing secretion of IL-18, proteases degrading IL-18, once it has been synthesized, inhibitors of proteases cleaving pro-IL-18 in order to generate mature IL-18, such as inhibitors of caspase-1, and the like.

An inhibitor of IL-18 action can be an IL-18 antagonist, for example. Antagonists can either bind to or sequester the IL-18 molecule itself with sufficient affinity and specificity to partially or substantially neutralise the IL-18 or IL-18 binding site(s) responsible for IL-18 binding to its ligands (like, e.g. to its receptors). An antagonist may also inhibit the IL-18 signalling pathway, which is activated within the cells upon IL-18/receptor binding.

Inhibitors of IL-18 action may be also soluble IL-18 receptors or molecules mimicking the receptors, or agents blocking the IL-18 receptors, or IL-18 antibodies, such as polyclonal or monoclonal antibodies, or any other agent or molecule preventing the binding of IL-18 to its targets, thus diminishing or preventing triggering of the intra- or extracellular reactions mediated by IL-18.

According to the first aspect of the present invention, inhibitors or IL-18 are used for the manufacture of a medicament for treatment and/or prevention of liver injury. Preferably, the invention relates to the use of an IL-18 inhibitor for the manufacture of a medicament for treatment and/or prevention of acute and chronic liver diseases, and more preferably, alcoholic hepatitis, viral hepatitis, immune hepatitis, fulminant hepatitis, liver cirrhosis, and primary biliary cirrhosis.

The term liver injury, or liver disease, a used herein, comprises a variety of different pathological conditions. Several of the conditions contemplated in the present invention have been explained in detail in the “Background of the invention” above. Further liver diseases which can be treated and/or prevented according to the invention comprise, for example, pyrogenic liver absess. It is also called bacterial liver, and it is a pus-producing cavity within the liver. The causes of a liver abscess are multiple. It can develop from an abdominal infection such as appendicitis, diverticulitis, or a perforated bowel; an infection in the blood; an infection from the biliary (liver secretion) tract; or trauma when a bruised liver becomes infected. The most common organisms causing liver abscess are Escherichia coli, Proteus vulgaris, and Enterobacter aerogenes. The incidence is 1 out of 10,000 people.

Alcoholic liver diseases can be treated and/or prevented using IL-18 inhibitors according to the invention. They comprise acute or chronic inflammation of the liver induced by alcohol abuse. Alcoholic hepatitis usually occurs after years of excessive drinking. The longer the duration of alcohol use and the larger the consumption of alcohol, the greater the probability of developing liver disease. Malnutrition develops as a result of empty calories from alcohol, reduced appetite, and malabsorption (inadequate absorption of nutrients from the intestinal tract). Malnutrition contributes to liver disease. The toxicity of ethanol to the liver, individual susceptibility to alcohol-induced liver disease, and genetic factors also contribute to the development of alcoholic liver disease.

In accordance with the present invention, liver cirrhosis can be treated and/or prevented using IL-18 inhibitors. Cirrhosis is a chronic liver disease which causes damage to liver tissue, scarring of the liver (fibrosis; nodular regeneration), progressive decrease in liver function, excessive fluid in the abdomen (ascites), bleeding disorders (coagulopathy), increased pressure in the blood vessels (portal hypertension), and brain function disorders (hepatic encephalopathy). The damaged and scarred liver becomes unable to adequately remove waste products (toxins) from the blood, and the formation of scar tissue leads to increased pressure (portal hypertension) in the veins between the intestines and spleen to the liver. Excessive alcohol use is the leading cause of cirrhosis. Other causes include infections (such as hepatitis), diseases and defects of the bile drainage system (such as biliary stenosis or obstruction), cystic fibrosis, and increased iron and copper absorption.

The type of cirrhosis depends on the cause of the disease. Complications of cirrhosis can be severe. In the U.S. cirrhosis is the 9th leading cause of death. Neurological problems (such as hepatic encephalopathy) can develop. Increased fluid collection in the abdominal cavity (ascites) is caused by decreased body protein, increased sodium, and increased pressure within the liver's blood vessels (portal hypertension). Portal hypertension can cause increased pressure, size, and fullness in the blood vessels in the esophagus (esophageal varices). Problems with bleeding and clotting can occur. The increased pressures within the blood vessels and the problems with blood clotting can increase the possibility of severe and life-threatening hemorrhage.

A further disorder meant to be encompassed by the term “liver injury” according to the present invention is autoimmune hepatitis. It is an inflammation of the liver caused by interaction with the immune system. Autoimmune hepatitis is a type of chronic active hepatitis. Cellular immune reactions may be a cause of chronic active hepatitis. A variety of circulating autoantibodies can be found in the blood of patients with chronic active hepatitis. Other autoimmune diseases may be associated with chronic active hepatitis, or may occur in the relatives of patients with chronic active hepatitis. These diseases are thyroiditis, diabetes mellitus, ulcerative colitis, Coombs-positive hemolytic anemia, proliferative glomerulonephritis, and Sjogren's syndrome. Risk factors may include these diseases, or risk factors associated with chronic active hepatitis. The incidence is 4 out of 10,000 people.

Biliary atresia is a further disorder within the scope of the term “liver injury”. It is an obstruction of the bile ducts caused by their failure to develop normally before birth (in utero). Biliary atresia is caused by the abnormal and inadequate development of the bile ducts inside or outside the liver. The purpose of the biliary system is to remove waste products from the liver, and to carry bile salts necessary for fat digestion to the small intestine. In this condition, bile flow from the liver to the gallbladder is blocked. This can lead to liver damage and cirrhosis of the liver, which, if not treated, is eventually fatal.

According to the invention, IL-18 inhibitors are also used for the manufacture of a medicament for treatment and/or prevention of chronic acitve hepatitis, also called chronic aggressive hepatitis. It is a continuing inflammation of the liver that damages the liver cells. Causes of chronic active hepatitis include viral infection, drug reaction/ingestion, metabolic disorders, or autoimmune diseases. There may also be no apparent cause. The disease is characterized by necrosis or death of liver cells, active inflammation, and fibrosis that may lead to liver failure, cirrhosis, and death. The incidence is 1 out of 10,000 people. Risk factors are autoimmune diseases, previous infection with hepatitis C, or a positive hepatitis A or hepatitis B antigen for over 6 months.

Chronic persistent hepatitis is a mild, nonprogressive form of liver inflammation, and it is also a disease encompassed by the term “liver injury” according to the present invention.

According to the invention, IL-18 inhibitors are also used for the manufacture of a medicament for treatment and/or prevention of primary biliary cirrhosis (PBC). PBC is an inflammatory condition resulting from obstruction of the flow of bile in the liver, causing damage to the liver cells. Bile ducts within the liver become inflamed due to unknown cause. The disease affects middle-aged women most frequently. The onset of symptoms is gradual, with itching skin as the first symptom. Inflammation of the bile ducts within the liver occurs. Eventually, liver cirrhosis develops. The disease may be associated with autoimmune disorders. The incidence is 8 out of 100,000 people. The IL-18 inhibitors contemplated herein may also be used for the treatment of acute hepatic poisoning, e.g. caused by a high amount of paracetamol. Such an acute hepatic poisoning may be due to an overdose of paracetamol, be it accidental or on purpose.

As shown in the examples below, the inventors of the present invention have surprisingly found that IL-18 inhibitors are particularly effective in the prevention and treatment of fulminant hepatitis (acute hepatitis). Therefore, the invention preferably relates to the prevention and/or or treatment of fulminant hepatitis.

According to the second aspect of the present invention, IL-18 inhibitors are used for the manufacture of a medicament for treatment and/or prevention of arthritis.

The term “arthritis” as used herein includes all different types of arthritis and arthritic conditions, both acute and chronic arthritis, as defined for example in the Homepage of the Department of Orthopaedics of the University of Washington on Arthritis (www.orthop.washington.edu). Examples for arthritic conditions are ankylosing spondylitis, back pain, carpal deposition syndrome, Ehlers-Danlos-Syndrome, gout, juvenile arthritis, lupus erythematosus, myositis, osteogenesis imperfecta, osteoporosis, polyartheritis; polymyositis, psoriatic arthritis, Reiter's syndrome, scleroderma, arthritis with bowel disease, Behcets's disease, children's arthritis, degenerative joint disease, fibromyalgia, infectious arthritis, Lyme disease, Marfan syndrome, osteoarthritis, osteonecrosis, Pagets Disease, Polymyalgia rheumatica, pseudogout, refelx sympathetic dystrophy, rheumatoid arthritis, rheumatism, Sjogren's syndrome, familial adenomatous polyposis and the like.

Preferably, according to the invention, inhibitors of IL-18 are provided for treatment and/or prevention of inflammatory arthritis. Inflammatory arthritis is classified as a chronic arthritis, according to the persistent, continuous or recurring course of the disease.

In a preferred embodiment of the invention, the inflammatory arthritis is rheumatoid arthritis (RA). RA causes inflammation in the lining of the joints (the synovial membrane, a one cell layer epithelium) and/or internal organs. The disease tends to persist for many years, typically affects many different joints throughout the body and ultimately can cause damage to cartilage, bone, tendons, and ligaments. The joints that may be affected by RA are the joints located in the neck, shoulders, elbows, hips, wrists, hands, knees, ankles and feet, for example. In many cases, the joints are inflamed in a symmetrical pattern in RA.

RA is prevalent in about 1% of the population in the United States, being distributed within all ethnic groups and ages. It occurs all over the world, and women outnumber men by 3 to 1 among those having RA.

As shown in the examples below, an inhibitor of IL-18 has been proven to exhibit a highly efficacious beneficial effect on cartilage erosion. The invention therefore further relates to the use of an inhibitor of IL-18 in the manufacture of a medicament for treatment and/or prevention of cartilage destruction, i.e. to the use of an IL-18 inhibitor as a chondroprotective agent. The IL-18 inhibitor may be used in any condition in which cartilage destruction or erosion occurs. Cartilage destruction is the progressive decline in the structural integrity of joint articular cartilage. It occurs for example in conditions affecting articular cartilage such as rheumatoid arthritis, juvenile rheumatoid arthritis, or osteoarthritis, but also in infectious synovitis, for instance.

The third aspect of the present nvention relates to the use of an IL-18 inhibitor for the manufacture of a medicament for the treatment and/or prevention of an inflammatory bowel disease. It is based on the finding that an inhibitor of IL-18 is upregulated in inflamed mucosa of CD patients. It is further based on the finding that the administration of different inhibitors of IL-18 have a protective effect in a murine model of colitis.

The upregulation of IL-18 in diseased mucosa from CD patients had already been described in the art (Monteleone, et al. 1999; Pizarro, et al. 1999).

After having demonstrated the presence of high amounts of IL-18BP in inflamed regions of the intestinal mucosa according to the invention, it was even more surprising to find a pronounced beneficial effect of IL-18BP administered systemically on the development and symptoms of colitis in an animal model. Although IL-18BP is already upregulated endogenously in the gut of CD patients, as demonstrated in the examples below, the amount of IL-18BP the body is capable of producing, does not seem to be sufficient to fight the disease.

Preferably, according to the invention the inflammatory bowel disease is Crohn's disease or ulcerative colitis.

In a preferred embodiment of the present invention, the inhibitor of IL-18 is selected from inhibitors of caspase-1 (ICE), antibodies directed against IL-18, antibodies directed against any of the IL-18 receptor subunits, inhibitors of the IL-18 signalling pathway, antagonists of IL-18 which compete with IL-18 and block the IL-18 receptor, and IL-18 binding proteins, isoforms, muteins, fused proteins, functional derivatives, active fractions or circularly permutated derivatives thereof having the same activity.

The term “IL-18 binding proteins” is used herein synonymously with “IL18BP”. It comprises IL-18 binding proteins as defined in WO 99/09063 or in Novick et al., 1999, including splice variants and/or isoforms of IL-18 binding proteins, as defined in Kim et al., 2000. In particular, human isoforms a and c of IL-18BP are useful in accordance with the presence invention. The proteins useful according to the present invention may be glycosylated or non-glycosylated, they may be derived from natural sources, such as urine, or they may preferably be produced recombinantly. Recombinant expression may be carried out in prokaryotic expression systems like E. coli, or in eukaryotic, and preferably in mammalian, expression systems.

As used herein the term “muteins” refers to analogs of an IL-18BP, or analogs of a viral IL-18BP, in which one or more of the amino acid residues of a natural IL-18BP or viral IL-18BP are replaced by different amino acid residues, or are deleted, or one or more amino acid residues are added to the natural sequence of an IL-18BP, or a viral IL-18BP, without changing considerably the activity of the resulting products as compared with the wild type IL-18BP or viral IL-18BP. These muteins are prepared by known synthesis and/or by site-directed mutagenesis techniques, or any other known technique suitable therefor.

Any such mutein preferably has a sequence of amino acids sufficiently duplicative of that of an IL-18BP, or sufficiently duplicative of a viral IL-18BP, such as to have substantially similar activity to IL-18BP. One activity of IL-18BP is its capability of binding IL-18. As long as the mutein has substantial binding activity to IL-18, it can be used in the purification of IL-18, such as by means of affinity chromatography, and thus can be considered to have substantially similar activity to IL-18BP. Thus, it can be determined whether any given mutein has substantially the same activity as IL-18BP by means of routine experimentation comprising subjecting such a mutein, e.g., to a simple sandwich competition assay to determine whether or not it binds to an appropriately labeled IL-18, such as radioimmunoassay or ELISA assay.

Muteins of IL-18BP polypeptides or muteins of viral IL-18BPs, which can be used in accordance with the present invention, or nucleic acid coding therefor, include a finite set of substantially corresponding sequences as substitution peptides or polynucleotides which can be routinely obtained by one of ordinary skill in the art, without undue experimentation, based on the teachings and guidance presented herein.

Preferred changes for muteins in accordance with the present invention are what are known as “conservative” substitutions. Conservative amino acid substitutions of IL-18BP polypeptides or proteins or viral IL-18BPs, may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g., under thirty, and preferably under ten, and do not remove or displace amino acids which are critical to a functional conformation, e.g., cysteine residues. Proteins and muteins produced by such deletions and/or insertions come within the purview of the present invention.

Preferably, the synonymous amino acid groups are those defined in Table I. More preferably, the synonymous amino acid groups are those defined in Table II; and most preferably the synonymous amino acid groups are those defined in Table III.

TABLE I
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE II
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE III
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Met |

Examples of production of amino acid substitutions in proteins which can be used for obtaining muteins of IL-18BP polypeptides or proteins, or muteins of viral IL-18BPs, for use in the present invention include any known method steps, such as presented in U.S. Pat. Nos. RE 33,653, 4,959,314, 4,588,585 and 4,737,462, to Mark et al; U.S. Pat. No. 5,116,943 to Koths et al., U.S. Pat. No. 4,965,195 to Namen et al; U.S. Pat. No. 4,879,111 to Chong et al; and U.S. Pat. No. 5,017,691 to Lee et al; and lysine substituted proteins presented in U.S. Pat. No. 4,904,584 (Shaw et al).

The term “fused protein” refers to a polypeptide comprising an IL-18BP, or a viral IL-18BP, or a mutein or fragment thereof, fused with another protein, which, e.g., has an extended residence time in body fluids. An IL-18BP or a viral IL-18BP, may thus be fused to another protein, polypeptide or the like, e.g., an immunoglobulin or a fragment thereof.

“Functional derivatives” as used herein cover derivatives of IL-18BPs or a viral IL-18BP, and their muteins and fused proteins, which may be prepared from the functional groups which occur as side chains on the residues or the N- or C-terminal groups, by means known in the art, and are included in the invention as long as they remain pharmaceutically acceptable, i.e. they do not destroy the activity of the protein which is substantially similar to the activity of IL-18BP, or viral IL-18BPs, and do not confer toxic properties on compositions containing it.

These derivatives may, for example, include polyethylene glycol side-chains, which may mask antigenic sites and extend the residence of an IL-18BP or a viral IL-18BP in body fluids. Other derivatives include aliphatic esters of the carboxyl groups, amides of the carboxyl groups by reaction with ammonia or with primary or secondary amines, N-acyl derivatives of free amino groups of the amino acid residues formed with acyl moieties (e.g. alkanoyl or carbocyclic aroyl groups) or O-acyl derivatives of free hydroxyl groups (for example that of seryl or threonyl residues) formed with acyl moieties.

As “active fractions” of an IL-18BP, or a viral IL-18BP, muteins and fused proteins, the present invention covers any fragment or precursors of the polypeptide chain of the protein molecule alone or together with associated molecules or residues linked thereto, e.g., sugar or phosphate residues, or aggregates of the protein molecule or the sugar residues by themselves, provided said fraction has substantially similar activity to IL-18BP.

In a further preferred embodiment of the invention, the inhibitor of IL-18 is an IL-18 antibody. Anti-IL-18 antibodies may be polyclonal or monoclonal, chimeric, humanised, or even fully human. Recombinant antibodies and fragments thereof are characterised by high affinity binding to IL-18 in vivo and low toxicity. The antibodies which can be used in the invention are characterised by their ability to treat patients for a period sufficient to have good to excellent regression or alleviation of the pathogenic condition or any symptom or group of symptoms related to a pathogenic condition, and a low toxicity.

Neutralising antibodies are readily raised in animals such as rabbits, goat or mice by immunisation with IL-18. Immunised mice are particularly useful for providing sources of B cells for the manufacture of hybridomas, which in turn are cultured to produce large quantities of anti-IL-18 monoclonal antibodies.

Chimeric antibodies are immunoglobulin molecules characterised by two or more segments or portions derived from different animal species. Generally, the variable region of the chimeric antibody is derived from a non-human mammalian antibody, such as murine monoclonal antibody, and the immunoglobulin constant region is derived from a human immunoglobulin molecule. Preferably, both regions and the combination have low immunogenicity as routinely determined (Elliott et al., 1994). Humanised antibodies are immunoglobulin molecules created by genetic engineering techniques in which the murine constant regions are replaced with human counterparts while retaining the murine antigen binding regions. The resulting mouse-human chimeric antibody preferably have reduced immunogenicity and improved pharmacokinetics in humans (Knight et al., 1993).

Thus, in a further preferred embodiment, IL-18 antibody is a humanised IL-18 antibody. Preferred examples of humanized anti-IL-18 antibodies are described in the European Patent Application EP 0 974 600, for example.

In yet a further preferred embodiment, the IL-18 antibody is fully human. The technology for producing human antibodies is described in detail e.g. in WO00/76310, WO99/53049, U.S. Pat. No. 6,162,963 or AU5336100. Fully human antibodies are preferably recombinant antibodies, produced in transgenic animals, e.g. xenomice, comprising all or parts of functional human Ig loci.

In a highly preferred embodiment of the present invention, the inhibitor of IL-18 is a IL-18BP, or an isoform, a mutein, fused protein, functional derivative, active fraction or circularly permutated derivative thereof. These isoforms, muteins, fused proteins or functional derivatives retain the biological activity of IL-18BP, in particular the binding to IL-18, and preferably have essentially at least an activity similar to IL-18BP. Ideally, such proteins have a biological activity which is even increased in comparison to unmodified IL-18BP. Preferred active fractions have an activity which is better than the activity of IL-18BP, or which have further advantages, like a better stability or a lower toxicity or immunogenicity, or they are easier to produce in large quantities, or easier to purify.

The sequences of IL-18BP and its splice variants/isoforms can be taken from WO99/09063 or from Novick et al., 1999, as well as from Kim et al., 2000.

Functional derivatives of IL-18BP may be conjugated to polymers in order to improve the properties of the protein, such as the stability, half-life, bioavailability, tolerance by the human body, or immunogenicity. To achieve this goal, IL18-BP may be linked e.g. to Polyethlyenglycol (PEG). PEGylation may be carried out by known methods, described in WO 92/13095, for example.

Therefore, in a preferred embodiment of the present invention, IL-18BP is PEGylated.

In a further preferred embodiment of the invention, the inhibitor of IL-18 is a fused protein comprising all or part of an IL-18 binding protein, which is fused to all or part of an immunoglobulin. The person skilled in the art will understand that the resulting fusion protein retains the biological activity of IL-18BP, in particular the binding to IL-18. The fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the IL-18BP sequence and the immunoglobulin sequence. The resulting fusion protein has improved properties, such as an extended residence time in body fluids (half-life), increased specific activity, increased expression level, or the purification of the fusion protein is facilitated.

In a preferred embodiment, IL-18BP is fused to the constant region of an Ig molecule. Preferably, it is fused to heavy chain regions, like the CH2 and CH3 domains of human IgG1, for example. The generation of specific fusion proteins comprising IL-18BP and a portion of an immunoglobulin are described in example 11 of WP99/09063, for example. Other isoforms of Ig molecules are also suitable for the generation of fusion proteins according to the present invention, such as isoforms IgG₂ or IgG₄, or other Ig classes, like IgM or IgA, for example. Fusion proteins may be monomeric or multimeric, hetero- or homomultimeric.

Interferons are predominantly known for inhibitory effects on viral replication and cellular proliferation. Interferon-γ, for example, plays an important role in promoting immune and inflammatory responses. Interferon β (IFN-β, an interferon type I), is said to play an anti-inflammatory role. Studies published by Triantaphyllopoulos et al (1999) indicated that IFN-β has a beneficial effect in the therapy of rheumatoid arthritis, as shown in a mouse model of the disease, the collagen-induced arthritis (CIA) model. This beneficial effect of IFN-β was confirmed in the examples below.

The invention also relates to the use of a combination of an inhibitor of IL-18 and an interferon in the manufacture of a medicament for the treatment of arthritis, in particular rheumatoid arthritis.

Interferons may also be conjugated to polymers in order to improve the stability of the proteins. A conjugate between Interferon β and the polyol Polyethlyenglycol (PEG) has been described in WO99/55377, for instance.

In another preferred embodiment of the invention, the interferon is Interferon-β (IFN-β), and more preferably IFN-β 1a.

The inhibitor of IL-18 production and/or action is preferably used simultaneously, sequentially, or separately with the interferon.

In yet a further embodiment of the invention, an inhibitor of IL-18 is used in combination with a TNF antagonist. TNF antagonists exert their activity in several ways. First, antagonists can bind to or sequester the TNF molecule itself with sufficient affinity and specificity to partially or substantially neutralise the TNF epitope or epitopes responsible for TNF receptor binding (hereinafter termed “sequestering antagonists”). A sequestering antagonist may be, for example, an antibody directed against TNF.

Alternatively, TNF antagonists can inhibit the TNF signalling pathway activated by the cell surface receptor after TNF binding (hereinafter termed “signalling antagonists”). Both groups of antagonists are useful, either alone or together, in combination with an IL-18 inhibitor, in the therapy of arthritis, in particular rheumatoid arthritis.

TNF antagonists are easily identified and evaluated by routine screening of candidates for their effect on the activity of native TNF on susceptible cell lines in vitro, for example human B cells, in which TNF causes proliferation and immunoglobulin secretion. The assay contains TNF formulation at varying dilutions of candidate antagonist, e.g. from 0.1 to 100 times the molar amount of TNF used in the assay, and controls with no TNF or only antagonist (Tucci et al., 1992).

Sequestering antagonists are the preferred TNF antagonists to be used according to the present invention. Amongst sequestering antagonists, those polypeptides that bind TNF with high affinity and possess low immunogenicity are preferred. Soluble TNF receptor molecules and neutralising antibodies to TNF are particularly preferred. For example, soluble TNF-RI and TNF-RII are useful in the present invention. Truncated forms of these receptors, comprising the extracellular domains of the receptors or functional portions thereof, are more particularly preferred antagonists according to the present invention. Truncated soluble TNF type-I and type-II receptors are described in EP914431, for example.

Truncated forms of the TNF receptors are soluble and have been detected in urine and serum as 30 kDa and 40 kDa TNF inhibitory binding proteins, which are called TBPI and TBPII, respectively (Engelmann et al., 1990). The simultaneous, sequential, or separate use of the IL-18 inhibitor with the TNF antagonist and/or an Interferon is preferred, according to the invention.

According to the invention, TBP I and TBPII are preferred TNF antagonists to be used in combination with an IL-18 inhibitor. Derivatives, fragments, regions and biologically active portions of the receptor molecules functionally resemble the receptor molecules that can also be used in the present invention. Such biologically active equivalent or derivative of the receptor molecule refers to the portion of the polypeptide, or of the sequence encoding the receptor molecule, that is of sufficient size and able to bind TNF with such an affinity that the interaction with the membrane-bound TNF receptor is inhibited or blocked.

In a further preferred embodiment, human soluble TNF-RI (TBPI) is the TNF antagonist to be used according to the invention. The natural and recombinant soluble TNF receptor molecules and methods of their production have been described in the European Patents EP 308 378, EP 398 327 and EP 433 900.

The IL-18 inhibitor can be used simultaneously, sequentially or separately with the TNF inhibitor. Advantageously, a combination of an IL-18 antibody or antiserum and a soluble receptor of TNF, having TNF inhibiting activity, is used.

In a further preferred embodiment of the invention, the medicament further comprises a COX-inhibitor, preferably a COX-2 inhibitor. COX inhibitors are known in the art. Specific COX-2 inhibitors are disclosed in WO 01/00229, for example.

The invention further relates to the use of a combination of IL-18 inhibitors and/or interferons and/or TNF antagonists and/or COX-2 inhibitors. The combination is suitable for the for the treatment and/or prevention of arthritis, in particular rheumatoid arthritis, and for the treatment and/or prevention of liver injury and for the treatment and/or prevention of inflammatory bowel disease, in particular Crohn's disease and ulcerative cloitis. The active components may be used simultaneously, sequentially, or separately.

In a preferred embodiment of the present invention, the inhibitor of IL-18 is used in an amount of about 0.0001 to 10 mg/kg of body weight, or about 0.01 to 5 mg/kg of body weight or about 0.1 to 3 mg/kg of body weight or about 1 to 2 mg/kg of body weight. In yet a further preferred embodiment, the inhibitor of IL-18 is used in an amount of about 0.1 to 1000 μg/kg of body weight or 1 to 100 μg/kg of body weight or about 10 to 50 μg/kg of body weight.

The invention further relates to the use of an expression vector comprising the coding sequence of an inhibitor of IL-18 in the preparation of a medicament for the prevention and/or treatment of arthritic conditions or arthritis, in particular rheumatoid arthritis, for the treatment of liver injury, and for the treatment of inflammatory bowel disease. A gene therapeutical approach is thus used for treating and/or preventing the disease. Advantageously, the expression of the IL-18 inhibitor will then be in situ, thus efficiently blocking IL-18 directly in the tissue(s) or cells affected by the disease.

In order to treat and/or prevent arthritis, the gene therapy vector comprising the sequence of an inhibitor of IL-18 production and/or action may be injected directly into the diseased joint, for example, thus avoiding problems involved in systemic administration of gene therapy vectors, like dilution of the vectors, reaching and targetting of of the target cells or tissues, and of side effects.

The use of a vector for inducing and/or enhancing the endogenous production of an inhibitor of IL-18 in a cell normally silent for expression of an IL-18 inhibitor, or which expresses amounts of the inhibitor which are not sufficient, are also contemplated according to the invention. The vector may comprise regulatory sequences functional in the cells desired to express the inhibitor or IL-18. Such regulatory sequences may be promoters or enhancers, for example. The regulatory sequence may then be introduced into the right locus of the genome by homologous recombination, thus operably linking the regulatory sequence with the gene, the expression of which is required to be induced or enhanced. The technology is usually referred to as “endogenous gene activation” (EGA), and it is described e.g. in WO 91/09955.

It will be understood by the person skilled in the art that it is also possible to shut down IL-18 expression using the same technique, i.e. by introducing a negative regulation element, like e.g. a silencing element, into the gene locus of IL-18, thus leading to down-regulation or prevention of IL-18 expression. The person skilled in the art will understand that such down-regulation or silencing of IL-18 expression has the same effect as the use of an IL-18 inhibitor in order to prevent and/or treat disease.

The invention further relates to the use of a cell that has been genetically modified to produce an inhibitor of IL-18 in the manufacture of a medicament for the treatment and/or prevention of liver injury, arthritis or inflammatory bowel disease.

The invention further relates to pharmaceutical compositions, particularly useful for prevention and/or treatment of inflammatory arthritis, liver injury or inflammatory bowel disease, which comprise a therapeutically effective amount of an inhibiter of IL-18 and a therapeutically effective amount of an interferon. As inhibitor of IL-18, the composition may comprise caspase-1 inhibitors, antibodies against IL-18, antibodies against any of the IL-18 receptor subunits, inhibitors of the IL-18 signalling pathway, antagonists of IL-18 which compete with IL-18 and block the IL-18 receptor, and IL-18 binding proteins, isoforms, muteins, fused proteins, functional derivatives, active fractions or circularly permutated derivatives thereof having the same activity.

IL-18BP and its isoforms, muteins, fused proteins, functional derivatives, active fractions or circularly permutated derivatives as described above are the preferred active ingredients of the pharmaceutical compositions.

The interferon comprised in the pharmaceutical composition is preferably IFN-β.

In yet another preferred embodiment, the pharmaceutical composition comprises therapeutically effective amounts of an IL-18 inhibitor, optionally an interferon, and a TNF antagonist. The TNF antagonists may be antibodies neutralising TNF activity, or soluble truncated TNF receptor fragments, also called TBPI and TPBII. The pharmaceutical composition according to the invention may further comprise one or more COX inhibitors, preferably COX-2 inhibitors.

The definition of “pharmaceutically acceptable” is meant to encompass any carrier, which does not interfere with effectiveness of the biological activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

The active ingredients of the pharmaceutical composition according to the invention can be administered to an individual in a variety of ways. The routes of administration include intradermal, transdermal (e.g. in slow release formulations), intramuscular, intraperitoneal, intravenous, subcutaneous, oral, epidural, topical, and intranasal routes. Any other therapeutically efficacious route of administration can be used, for example absorption through epithelial or endothelial tissues or by gene therapy wherein a DNA molecule encoding the active agent is administered to the patient (e.g. via a vector) which causes the active agent to be expressed and secreted in vivo. In addition, the protein(s) according to the invention can be administered together with other components of biologically active agents such as pharmaceutically acceptable surfactants, excipients, carriers, diluents and vehicles.

For parenteral (e.g. intravenous, subcutaneous, intramuscular) administration, the active protein(s) can be formulated as a solution, suspension, emulsion or lyophilised powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The bioavailability of the active protein(s) according to the invention can also be ameliorated by using conjugation procedures which increase the half-life of the molecule in the human body, for example linking the molecule to polyethylenglycol, as described in the PCT Patent Application WO 92/13095.

The therapeutically effective amounts of the active protein(s) will be a function of many variables, including the type of antagonist, the affinity of the antagonist for IL-18, any residual cytotoxic activity exhibited by the antagonists, the route of administration, the clinical condition of the patient (including the desirability of maintaining a non-toxic level of endogenous IL-18 activity

A “therapeutically effective amount” is such that when administered, the IL-18 inhibitor results in inhibition of the biological activity of IL-18. The dosage administered, as single or multiple doses, to an individual will vary depending upon a variety of factors, including IL-18 inhibitor pharmacokinetic properties, the route of administration, patient conditions and characteristics (sex, age, body weight, health, size), extent of symptoms, concurrent treatments, frequency of treatment and the effect desired. Adjustment and manipulation of established dosage ranges are well within the ability of those skilled in the art, as well as in vitro and in vivo methods of determining the inhibition of IL-18 in an individual.

According to the invention, the inhibitor of IL-18 is used in an amount of about 0.0001 to 10 mg/kg or about 0.01 to 5 mg/kg or body weight, or about 0.01 to 5 mg/kg of body weight or about 0.1 to 3 mg/kg of body weight or about 1 to 2 mg/kg of body weight. Further preferred amounts of the IL-18 inhibitors are amounts of about 0.1 to 1000 μg/kg of body weight or about 1 to 100 μg/kg of body weight or about 10 to 50 μg/kg of body weight

The route of administration which is preferred according to the invention is administration by subcutaneous route. Intramuscular administration is further preferred according to the invention.

In further preferred embodiments, the inhibitor of IL-18 is administered daily or every other day.

The daily doses are usually given in divided doses or in sustained release form effective to obtain the desired results. Second or subsequent administrations can be performed at a dosage which is the same, less than or greater than the initial or previous dose administered to the individual. A second or subsequent administration can be administered during or prior to onset of the disease.

According to the invention, the IL-18 inhibitor can be administered prophylactically or therapeutically to an individual prior to, simultaneously or sequentially with other therapeutic regimens or agents (e.g. multiple drug regimens), in a therapeutically effective amount, in particular with an interferon and/or a TNF antagonist and/or a COX inhibitor. Active agents that are administered simultaneously with other therapeutic agents can be administered in the same or different compositions.

The invention further relates to a method for the preparation of a pharmaceutical composition comprising admixing an effective amount of an IL-18 inhibitor and/or an interferon and/or a TNF antagonist and/or a COX inhibitor with a pharmaceutically acceptable carrier.

Having now described the invention, it will be more readily understood by reference to the following examples that are provided by way of illustration and are not intended to be limiting of the present invention.

### EXAMPLES

PART I: Examples 1 to 8, relating to the use of IL-18 inhibitors in liver injury

### Example 1

### Production of IL-18BP-His Tag

Purified recombinant human IL18BP containing a his-tag (r-hIL-18BP-His tag) was produced in CHO cells. The production of recombinant proteins in eukaryotic cells is known by the person skilled in the art. Well known methods are available for constructing appropriate vectors, carrying DNA that codes for IL-18BP and suitable for transfection of eukaryotic cells in order to produce recombinant IL-18BP. For expression in cells, the DNA coding for IL-18BP (see, e.g. (Novick et al., 1999) is cut out and inserted into expression vectors suitable for transfection of cells. Alternatively, such DNA can be prepared by PCR with suitable sense and antisense primers. The resulting cDNA constructs are then inserted into appropriately constructed eukaryotic expression vectors by techniques well known in the art (Manaitis, 1982). The recombinant protein was purified to over 95% purity and found to be biologically active in-vitro and in-vivo with a high affinity to its ligand.

### Example 2

### Protective Effect of IL18BP Against Endotoxin-Induced Death in the Murine Model

A murine model was used to test whether IL18BP, an inhibitor of IL-18, would protect mice against a high dose of lipopolysaccharides (LPS). LPS elicits acute liver injury, followed by rapid death of the mice.

4 mg/kg of recombinant, human IL-18BP (rhIL18BPhis) containing a his-tag (resulting from recombinant production of the protein) was injected intraperitoneally (i.p). into C57BL/6 mice. 1 h later, 60 mg/kg LPS were injected (lethal doses). The survival of mice was compared to a group of animals who received LPS alone (no IL18BP).

Five out of 7 mice injected with rhIL-18BP-his survived the LPS injection in contrast to the control mice, in which all animals died within 3 days.

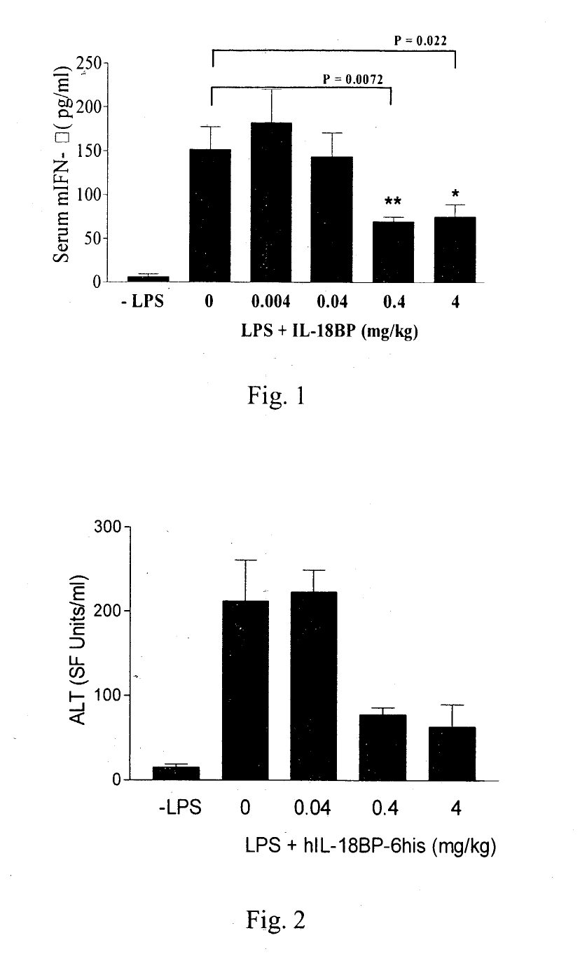

Blood samples were taken 5 h after the LPS injection in the absence or presence of increasing doses of rhIL-18BP-his and analyzed by ELISA for circulating IFN-γ (FIG. 1). 0.4 and 4 mg/kg rhIL-18BP induced a 2 fold reduction in serum IFN-γ. This inhibition was lost at lower doses of rhIL-18BP (0.004 and 0.4 mg/kg).

### Example 3

### IL18BP Has a Protective Effect Against Liver Injury in a Murine Model of Disease

A mouse model of fulminant hepatitis was used to test the effect of IL18BP. Mice develop acute liver injury when subjected to a sequential administration of Propionibacterium acnes (P. acnes) and lipopolysaccharide (LPS).

Mice were injected with increasing doses of rhIL-18BP-his (4; 0.4; 0.04; 0 mg/kg) at various times (1 h, 20 min, simultaneously) before the injection of LPS in C57BL/6 P. acnes sensitized mice. When rhIL-18BP-his was given i.p. at the same time as LPS, none of the mice survived and levels of circulating IFN-γ and TNF-α were unaffected. Surprisingly, rhIL-18BP (4 and 0.4 mg/kg) induced a 70% reduction of circulating Alanine aminotransferase (a marker of liver injury), as shown in FIG. 2.

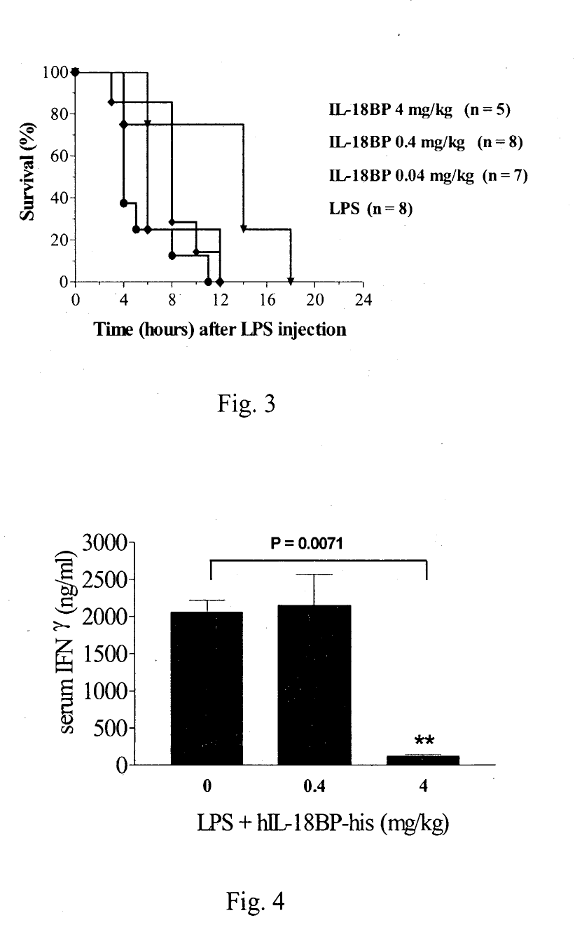

In addition to this, the survival of mice was monitored (FIG. 3): When rhIL-18BP was given i.p. 20 minutes before LPS, the two highest doses of Il-18BP (4 and 0.4 mg/kg) delayed the death of the mice by 10 h as compared to the control mice who received NaCl instead of IL-18BP.

The results of the measurement of serum IFN-γ levels are shown in FIG. 4. rhIL-18BP (4 mg/kg) inhibited 90% of circulating IFN-γ levels and 80% of circulating Alanine aminotransferase (not shown).

When rhIL-18BP-his was given 1 h before LPS, survival curves and levels of circulating IFN-γ were similar to what was observed when rhIL-18BP-his was given 20 min before LPS, but levels of circulating Alanine aminotransferase were unaffected (not shown).

In addition to this, murine liver tissue was analyzed by hematoxilin-eosine staining, as well as by tunnel microscopy. The livers of mice, in which severe hepatitis had been induced before, showed severe necrosis as compared to normal liver tissue. In contrast to this, liver tissue of mice treated with IL-18BP showed significantly less necrotic foci than untreated mice.

### Example 4

### Anti-IL-18 Antibodies Protect Against Lethal Endotoxemia

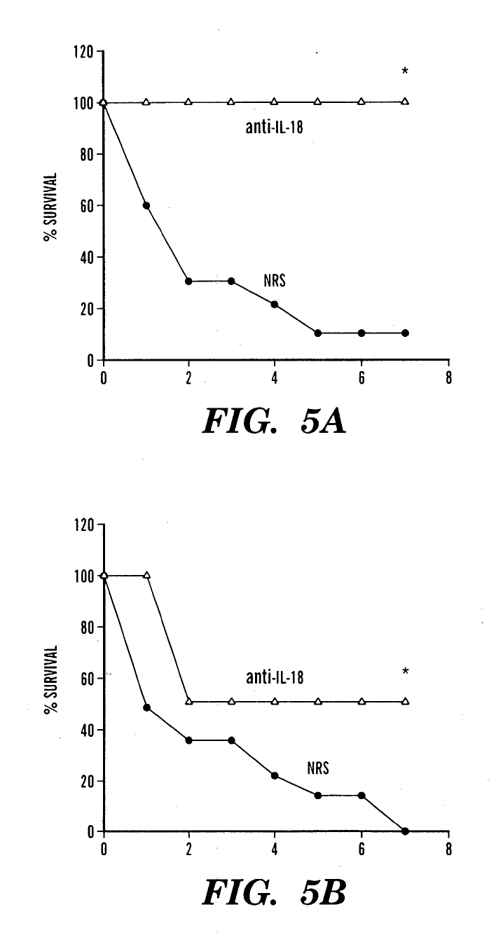

In order to evaluate, whether blockade of IL-18 with IL-18 antibodies would protect mice against lethal doses of bacterial lipopolysaccharides, C57BL/6J mice were first injected with a neutralizing rabbit anti-mouse IL-18 antibody (polyclonal) or normal rabbit serum (NDS) as a control. 30 min after antibody treatment, a lethal dosis of LPS derived either from E. coli (FIG. 5 A) or S. thyphimurium (FIG. 5 B) was injected. Experiments involved 10-12 mice/group, and were performed twice on two different occasions.

As shown in FIG. 5 A, treatment of the mice with the anti-IL-18 antiserum prevented the mortality induced by 40 mg/kg E. coli LPS. 100% of the mice survived after anti-IL-18 treatment vs. 10% survival in mice treated with normal rabbit serum (p<0.005).

FIG. 5 B shows that the antibody treated mice were also protected against S. typhimurium lethal effects (50% vs. 0% survival; p<0.05).

### Example 5

### Blockade of IL-18 and TNF-α Protects Mice from ConA- and PEA-induced Hepatotoxicity

Two experimental models of hepatotoxicity were used to evaluate the role of IL-18 and TNF-α in liver injury. Injection of Concanavalin A (Con A) and Pseudomonas aeruginosa (PEA) into mice both induce liver injury, and are models of T cell mediated hepatitis.

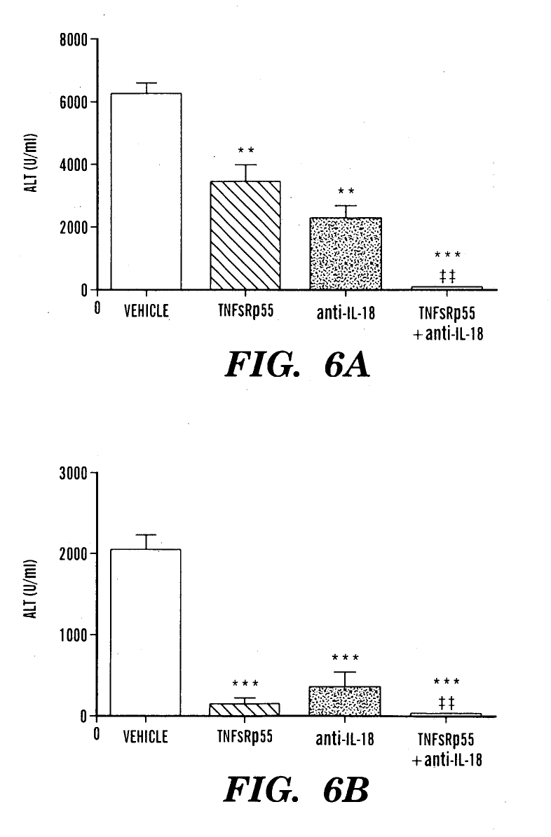

C57BL/6J mice were pretreated with an anti-IL-18 antiserum or a soluble TNF-α receptor, TNFsRp55. Serum Alanine aminotransferase (ALT) levels were measured as indicators of hepatic injury (FIG. 6).

As shown in FIG. 6 A, both IL-18 antiserum and soluble TNF-receptors significantly reduced ConA-induced serum ALT levels, as compared to a control injection of the vehicle alone (pyrogen free saline). A co-adminstration of soluble TNF receptor and IL-18 antserum led to a complete inhibition of Con-A induced liver injury.

As shown in FIG. 6 B, in PEA-injected mice, neutralization of either TNF-α inhibitors or anti-IL-18 antibodies resulted in 93% and 83% inhibition of serum ALT levels, respectively. A combined blockade of both resulted in 99% protection.

### Example 6

### Plasma Levels of IL-18-Binding Protein Are Elevated in Patients with Chronic Liver Disease

IL-18 BP plasma levels were measured in 133 patients with chronic liver disease (CLD) of varying etiologies and 31 healthy controls by a specific ELISA, using an IL-18BP monoclonal antibody.

Plasma levels of IL-18 BP were significantly higher in CLD patients (12.91±0.89 ng/ml; average±SEM) than in healthy subjects (4.96±0.43 ng/ml, p<0.001). Cirrhotic patients had signifcantly higher levels than patients with non-cirrhotic CLD (19.23±1.28 ng/ml, n=67, vs. 6.49±0.51 ng/ml, n=66, p<0.001). Patients with stage B of the Child-Pugh classification had higher levels of IL-18 BP than those with stage A (22.48±2.44 ng/ml vs. 9.57±1.25 ng/ml, p<0.001). However, there was no significant difference between Child B and C (22.48±2.44 ng/ml vs. 20.62±4.75 ng/ml, p=0.7). Plasma levels of IL-18 BP correlated positively with GOT, bilirubin and erythrocyte sedimentation rate. Negative correlation was found with prothrombin time.

In conclusion, the results show that IL-18 BP plasma levels are elevated in CLD and correlate with the severity of disease independent of the etiology of disease. Although an endogenous antagonist of the proinflammatory IL-18, increased levels of IL-18 BP seem not to be sufficient to counteract the overwhelming proinflammatory mediators in CLD.

### Example 7

### Inhibition of Alcoholic Hepatitis by IL-18BP

Four groups of rats (5 per group) are fed ethanol and a diet containing corn oil by intragastric infusion for 4 weeks. Dextrose isocalorically replaces ethanol in control rats. The rats are injected daily with mouse IL-18BP (1 mg/kg), or saline. Pathological analysis is performed on liver sections and measurements of liver enzymes in serum, TNF-α, Fas ligand and IFN-γ are taken. Necroinflammatory injury and expression of liver enzymes, TNF-α, Fas ligand, and IFN-γ are seen in the ethanol-fed rats that were injected with saline.

Rats injected with mouse IL-18BP are protected from necroinflammatory injury and the levels of liver enzymes, TNF-α, Fas ligand and IFN-γ are significantly reduced (>90%).

### Example 8

### Inhibition of Concanavalin A-Induced Hepatitis by IL-18BP

Balb/c mice are injected with 12 mg/kg Concanavalin A (Con A) with or without injection of murine IL-18BP (1 mg/kg), 2 h prior to Con A administration and then daily. Liver damage is evaluated by determining serum levels of liver enzymes, TNF-α, Fas ligand and IFN-γ. Hepatic histopathology is compared to mice treated with Concanavalin A only.

Pretreatment with IL-18BP significantly reduces serum levels of liver enzymes and TNF-α with no evidence of inflammation in histopathologic examination compared to control mice treated with Con A.

PART II: Examples 9 and 10 relating to the use of IL-18 inhibitors in arthritis

### Example 9

### Production of IL-18BP-His Tag

For the experiment described in detail in Example 10 below, recombinant human IL-18BP containing a his-tag of 6 residues (r-hIL-18BP-His tag) was produced in CHO cells and purified as described by Kim et al., 2000. The recombinant protein was purified to over 95% purity and found to be biologically active in-vitro and in-vivo with a high affinity to its ligand.

The production of recombinant proteins in other eukaryotic systems, with or without tags facilitating purification of the recombinant proteins, is known by the person skilled in the art. Well known methods are available for constructing appropriate vectors, carrying DNA that codes for IL-18BP and suitable for transfection of eukaryotic cells in order to produce recombinant IL-18BP. For expression in cells, the DNA coding for IL-18BP (see, e.g. Novick et al., 1999) is cut out from the cloning vector and inserted into expression vectors suitable for transfection of cells. Alternatively, such DNA can be prepared by PCR with suitable sense and antisense primers. The resulting cDNA constructs are then inserted into appropriately constructed eukaryotic expression vectors by techniques known in the art (Maniatis et al., 1982).

### Example 10

### Blockade of Endogenous IL-18 in a Murine Model of Arthritis

### Methods

Induction of collagen-induced arthritis (CIA)

CIA was induced in male DBA/1 mice (8-12 weeks old) by immunisation with native type II bovine collagen (CII) as previously described (Plater-Zyberk et al., 1995). From day 25 post-CII immunisation, mice were examined daily for onset of disease.

Treatment with rhIL-18BP-6his

Treatment of CII-immunised DBA/1 mice was started on the first appearance of clinical sign of disease. Recombinant, human IL-18BP containing a tag of 6 histidines (rh-IL-18BPa 6his) was used to neutralise endogenous IL18 in the collagen treated mice. rh-IL-18BP-6his was injected daily for 7 seven days at 5 different concentrations 10, 3, 1, 0.5, 0.25 mg/kg/injection intraperitoneally (i.p.). The placebo control mice received the vehicle only (0.9% NaCl).

Assessment of disease development

Clinical evaluation (clinical scores)

From first appearance of clinical symptoms, mice were examined every day by an investigator blinded to the treatment. Each limb was graded for disease severity (scores 0-3.5, max score=14/mouse). The progression of edema (inflammation) was measured on the first paws that showed signs of disease using precision calipers (Proctest 2T, Kroeplin Langenmesstechnik)

Disease progression was assessed daily for 8 days post-onset at which time all mice were sacrificed and paws collected for histopathological examination.

Histological assessment of cartilage erosions and microscopical inflammation

At termination of the experiments, i.e. at day 8 post-onset, mice were killed and the paw that first developed sign of disease was dissected away. Joints were fixed, decalcified and embedded in paraffin. Standard sections (5 to 7 μm) of the joints were made and stained with hematoxylin/eosin/Safranin O. Each joint was scored by 2 investigators unaware of the treatment protocol (no destruction of cartilage or bone=0; localised cartilage erosions=1-2; more extended erosions=3; general cartilage destruction and presence of bone erosions=4). The final scores of each mouse was the mean of the result on all the scored joints. Microsopical inflammation or synovitis was scored from 0 to 4, as follows: no inflammation=0; slight thickening of lining layer and/or some infiltrating cells in sublining layer=1-2; thickening of lining layer and/ more pronounced influx of cells of sublining layer=3; presence cells in the synovial space and synovium highly infiltrated with many inflammatory cells=4.

Determination of anti-collagen antibodies.

Antibodies against bovine type II collagen were examined by using an enzyme-linked immunosorbent assay (ELISA). Titers of IgG1 and IgG2a were measured. Briefly, plates were coated with 10 μg of bovine collagen and thereafter-nonspecific binding sites were blocked with 0.1 M ethanolamin (Sigma). Serial 1:2 dilutions of the sera were added followed by incubation with isotype specific goat anti-mouse peroxidase (Southern Biotechnology Associates, Birmingham, Ala., USA) and substrate (5-aminosalicyclic acid, Sigma). Plates were read at 492 nm. Titers were expressed as mean±SD dilution, which gives the half-maximal value.

IL-6 assays

Levels of IL-6 were determined by commercial ELISA (R&D systems, Minneapolis, Minn., USA). IL-6 bioactivity was determined by a proliferative assay using B9 cells. Briefly, 5×10³ B9-cells in 200 μl 5% FCS-RPMI 1640 medium per well were plated in a round-bottom microtitre plate and incubated for 3 days using human recombinant IL-6 (R&D systems, Minneapolis, Minn., USA) as standards. At the end of the incubation, 0.5 μCi of ³[H]thymidine (NEN-Dupont, Boston, Mass., USA) was added per well. Three hours later, cells were harvested and thymidine incorporation was determined. Detection limit for the IL-6 bioassay was 1 pg/ml.

Statistical analysis

Significance of differences was assessed by the Mann Whitney test using SigmaStat statistical analysis program and the GraphPad Prism program.

### Results

A mouse experimental model, CIA (collagen induced arthritis), was used for assessing the effectiveness of IL-18BP as an agent for the treatment of arthritis. Administration of collagen and incomplete Freund's adjuvant in DBA/1 mice induces the development of an erosive, inflammatory arthritis and represents an ideal opportunity to explore the therapeutic potential of IL-18BP. To this end, endogenous IL-18 was neutralised using IL-18BP and the effect on various pathogenic parameters was evaluated.

A dose-related study was performed. Three groups of collagen-induced arthritic DBA/1 mice were treated therapeutically (i.e. after onset of disease) with 5 doses of IL-18BP i.p. (intraperitoneal). IL-18BP at concentrations of 10, 3, 1, 0.5 or 0.25 mg/kg was administered at the first clinical sign of disease. Injection of physiological saline (sodium chloride, NaCl) was used as a control. In addition to this, 10000 IU of interferonβ (IFN-β) were administered i.p. to assess the effect of IFN in this experimental model of arthritis. The effect on disease severity was monitored by daily visual scoring of each individual paw as described above. The mice were sacrificed at day 8 post-onset.

The following values were measured:

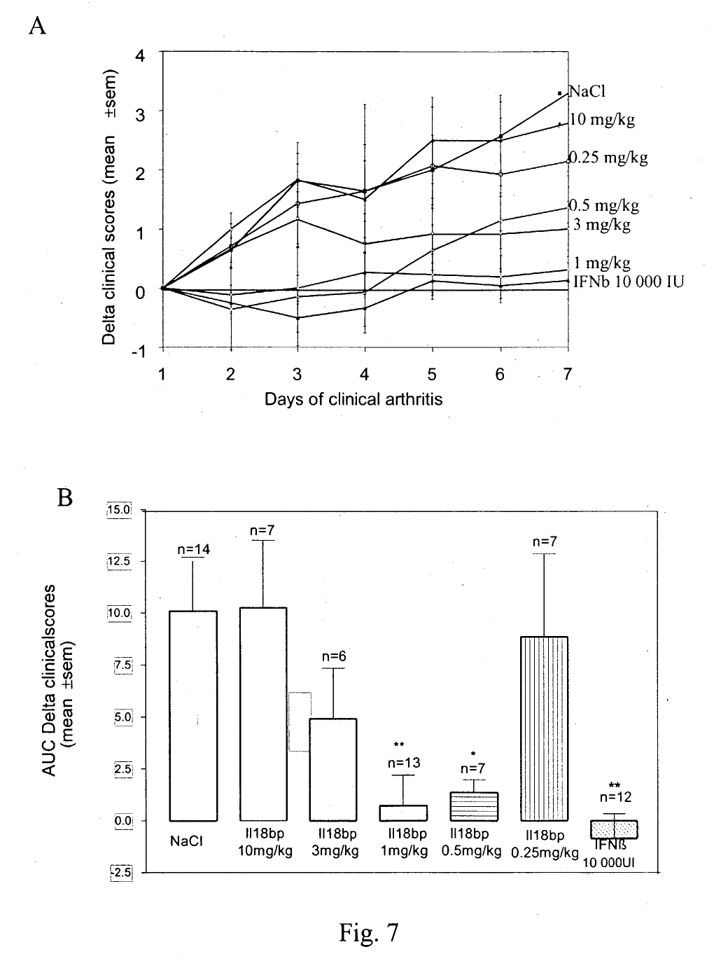

visual clinical scores (0-3.5 per paw) (FIGS. 7 A and B)

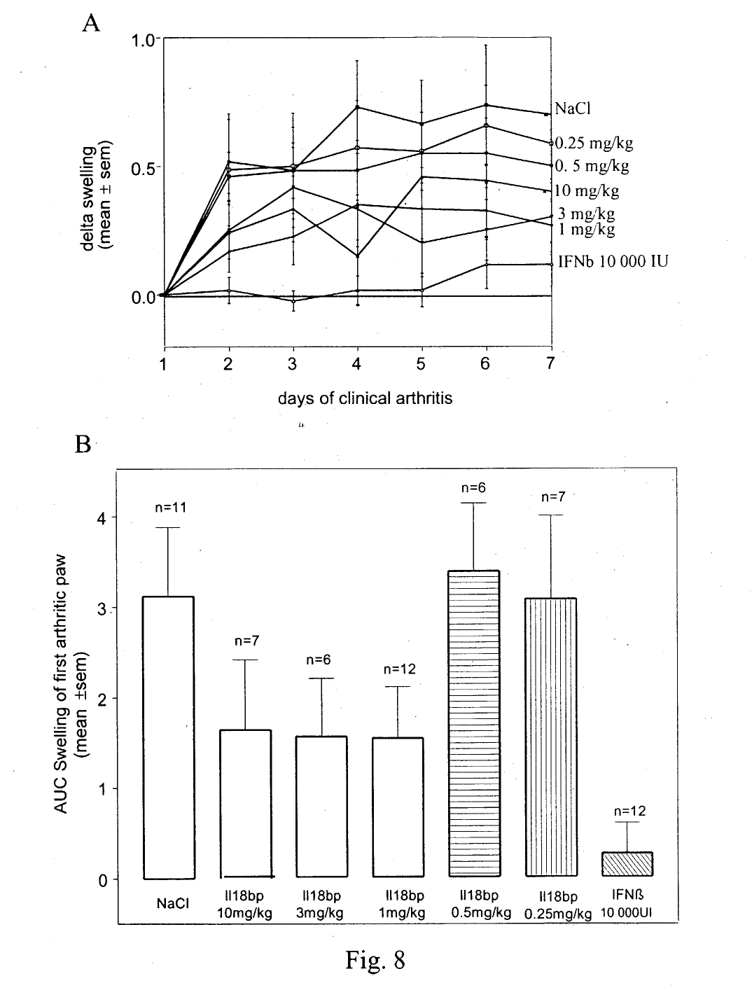

joint swelling/edema (in mm, measured with calipers) of first diseased paw, provided it was a hind paw (FIG. 8)

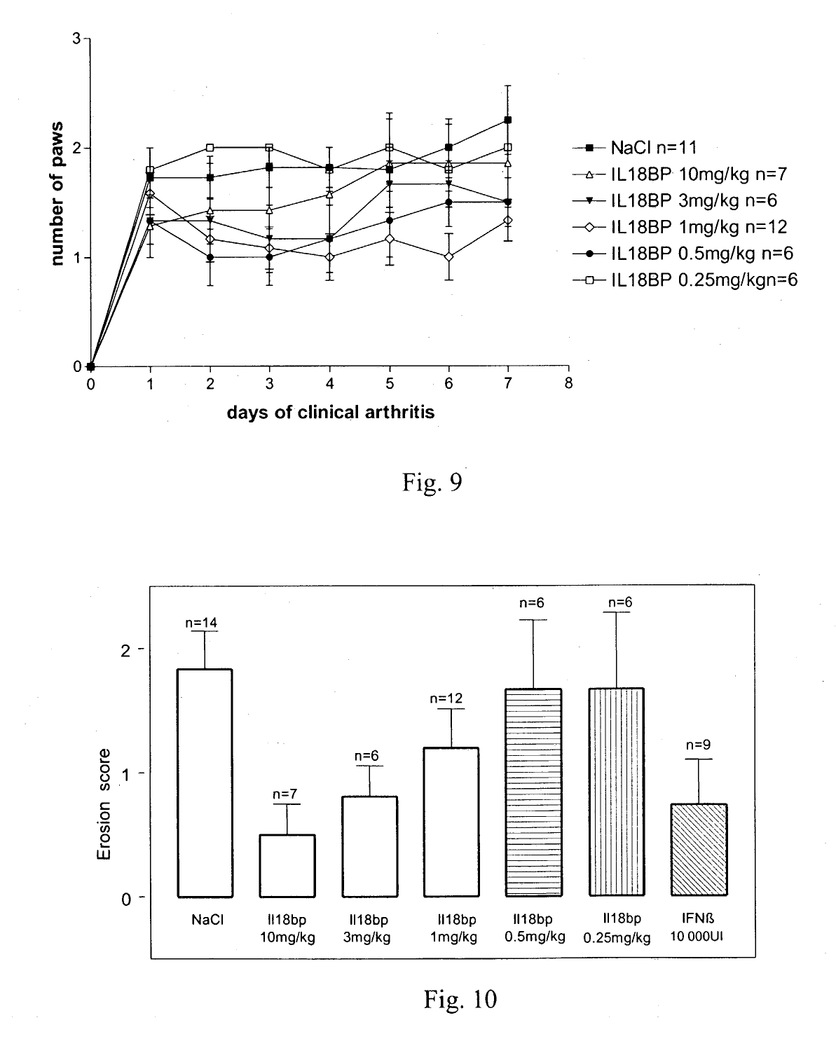

number of paws recruited into the disease (FIG. 9)

erosion scores of first diseased paw (0-4 cartilage destruction, FIG. 10).

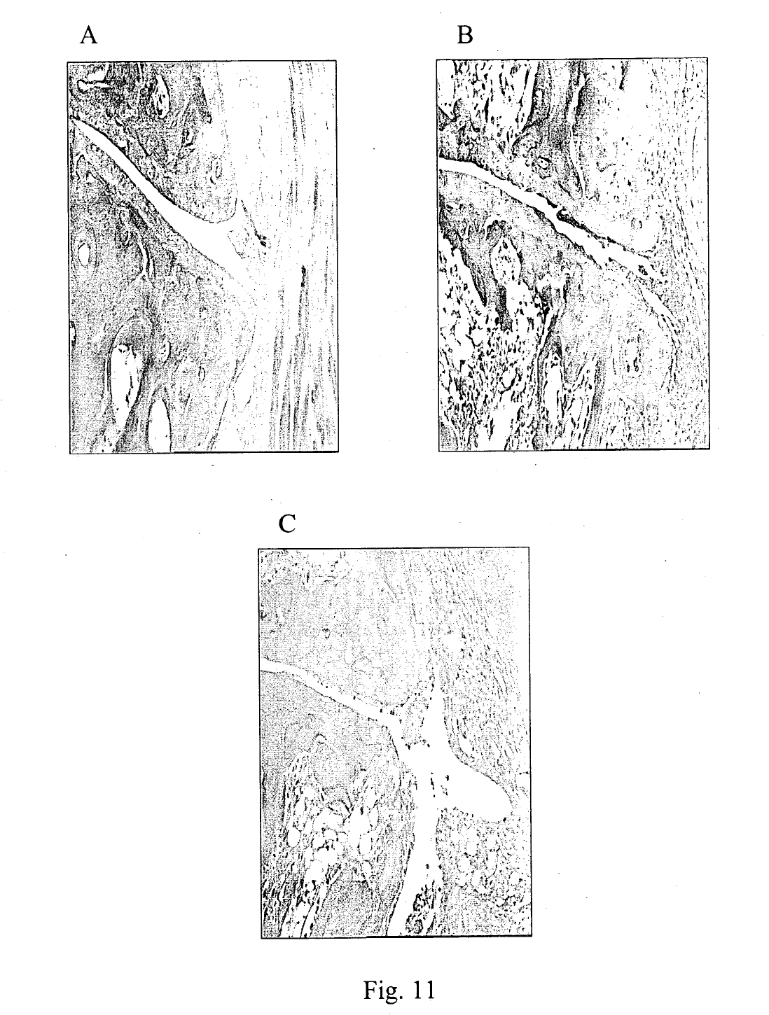

Histopathological analysis of the paw that first developed arthritis (FIG. 11)

Levels of anti-collagen type II antibodies (FIG. 12)

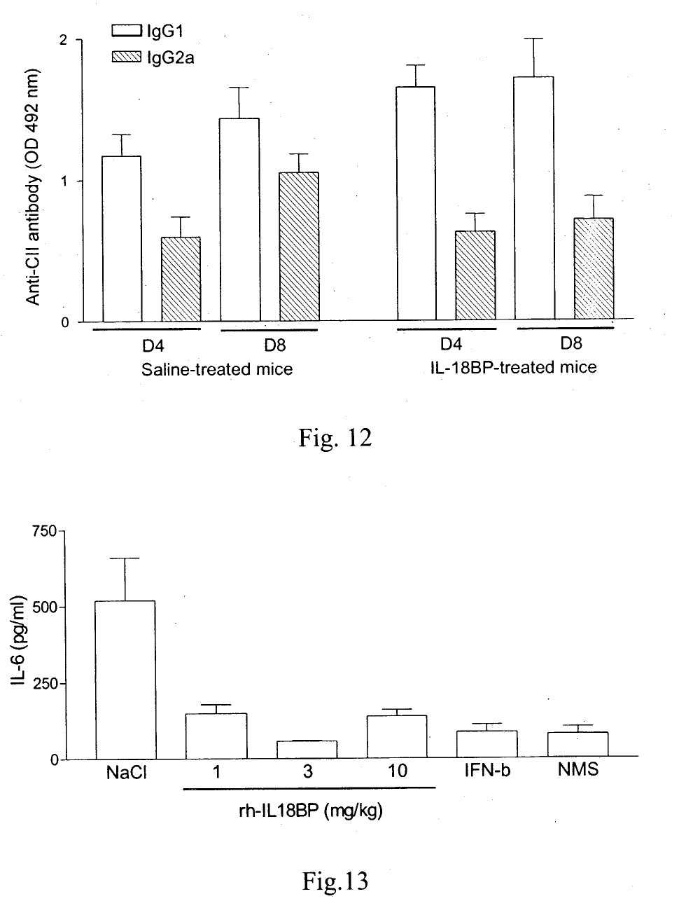

Levels of IL-6 (FIG. 13).

Clinical severity of disease

As shown in FIGS. 7 A and B, the clinical severity of disease was significantly diminished in the groups treated with 1 mg/ml (P<0.01) and 0.5 mg/ml (0.01<P<0.05) of rhIL-18BP. Mice receiving the low dose of rhIL-18BP (0.25 mg/kg) or the high dose of 10 mg/kg had a clinical score similar to the placebo group. The dose of 1 mg/kg of IL-18BP was approximately as effective as Interferon β (designated IFNb in FIG. 7 A).

Joint inflammation and paw swelling (edema)

Macroscopical inflammation (swelling) was studied by measuring paw edema from day 1 after onset of disease until day 8, the end of the experiment. The results are shown in FIGS. 8 A and B. The effective doses of IL-18BP were 1, 3 and 10 mg/kg. Administration of lower doses did not result in a beneficial effect on the swelling of paws. As shown in FIGS. 8 A and 8 B, lnterferon-β (IFNb) at a concentration of 10000 IU showed a beneficial effect on paw swelling.

Microscopioc synovitis was examined at the end of the experiment on histopathological sections and was expressed as scores (“synovitis score”). The results of inflammation (swelling) and synovitis score are summarised in Table 1. Whilst treatment with rhIL-18BP at dosages 1 and 3 mg/kg resulted in a trend towards reduction of swelling, treatment with any dosage of IL-18BP had only a limited effect on the inflammatory synovitis (Table 1).

TABLE 1
EFFECT OF IL-18BP TREATMENT
ON JOINT INFLAMMATION

| | | | Swelling | Synovitis score |
|---|---|---|---|---|
| Treatment | | | (AUC, mean ± sem) | (mean ± sem) |
| RhIL-18BP-6his | 3 | mg/kg | 1.55 ± 0.64 | 2.17 ± 0.5 |
| | 1 | mg/kg | 1.53 ± 0.60 | 1.98 ± 0.4 |
| | 0.5 | mg/kg | 3.38 ± 0.70 | 1.92 ± 0.5 |
| | 0.25 | mg/kg | 3.06 ± 0.90 | 2.08 ± 0.5 |
| Placebo | | | 3.11 ± 0.77 | 2.23 ± 0.3 |

FIG. 9 shows that the number of paws affected by the disease was diminished after administration of IL-18BP. In particular, therapeutic injections of IL-18BP at doses of 1 and 0.5 mg/kg reduced the number of paws recruited into the disease, demonstrating that blockade of IL-18 in vivo halts the spreading of arthritis to additional joints. Treatment with 1 and 0.5 mg/kg of IL-18BP even appears capable to reverting some of the arthritic joints to normality.

Protection from joint destruction

Treatment of mice with rhIL-18BP resulted in protection of joints from destruction (FIG. 10). A semi-quantitative scoring system demonstrated that bone erosion showed a dose-related protective effect that was significant at 10 and 3 mg/kg (P<0.05, FIG. 10). Mice receiving 1 mg/kg of rhIL-18BP presented less erosion than mice receiving vehicle only. No protection was observed at doses of 0.5 mg/kg and 0.25 mg/kg. Interestingly, the effect on joint protection at doses of 3 and 10 mg/kg IL-18BP were comparable to or even more pronounced than the beneficial effect of 10000 IU of Interferon β (IFN-β).

FIG. 11 shows the histology of a healthy (A) and a diseased (B) joint in comparison to a joint derived from an animal treated with IL-18BP (C). Sections were taken at the end of the experiment from those paws which first developed arthritis

The joint from an arthritic mouse showed severe destructive arthritis with cartilage depletion and erosions and numerous infiltrating cells in the inflamed synovium. In the joint from a mouse treated with rhIL-18BP, the cartilage appeared almost normal despite the presence of inflammatory cells in the synovial space. There was not only a higher amount of cartilage, but the cartilage has also a smoother appearance.

Anti-IL-18 treatment modulates levels of anti-type II collagen antibodies

CIA mice have elevated levels of IgG1 and IgG2a anti-type II collagen antibodies in the circulation. Antibodies of the isotype IgG1 are associated to TH2 mediated diseases, whereas antibodies of the isotype IgG2a and IgG2b are associated to TH1 mediated diseases. Arthritis is usually classified as a TH1 mediated disease.

Anti-type II collagen (CII) IgG1 and IgG2a antibody isotypes were determined in the sera of animals that were treated with IL-18BP (FIG. 12). Levels of anti-CII of the IgG isotypes IgG1 and IgG2a were not significantly modified by IL-18BP treatment at day 4 or 8 (D4, D8) of clinical disease. However, a 2.6 and 3.4 fold decrease in collagen-specific IgG1/IgG2a ratios was observed after 8 days of rhIL-18BP-treatment, at 1 and 3 mg/kg respectively. FIG. 12 shows the experiment in which 3 mg/kg were used. Essentially the same results were obtained using an amount of 1 mg/kg of IL-18BP. The reduced IgG1/IgG2a ratio of anti-CII antibodies indicate a diminished concentration of anti-type II collagen antibodies of the isotype IgG2a and an elevated concentration of anti-type II collagen antibodies of the isotype IgG1, suggesting that there is an shift towards TH2-mediated disease in this model of arthritis.

Reduction of IL-6 levels after IL-18 neutralisation

To gain insight into the effects of IL-18 blockade, IL-6 was measured in the sera of IL-18BP treated animals. FIG. 13 shows that the levels of bioactive IL-6 was significantly reduced in the animals having received IL-18BP treatment at all doses measured, i.e. at 1, 3 and 10 mg/kg as well as with Interferon-β (IFNb). Immunoactive levels of IL-6 measured in the sera of the animals treated with 3 mg/kg rhIL-18BP were significantly reduced (P<0.0023) as compared with saline-treated animals. IL-6 serum levels of diseased animals treated with 1, 3 or 10 mg of IL-18BP or 10000 IU of IFN-β were similar to normal mouse serum (NMS) derived from healthy animals, i.e. from those animals not having an inflammatory disease.

These findings demonstrate that IL-18 controls IL-6 levels during the onset of the disease. Since IL-6 is a marker of inflammation, these findings show that treatment of diseased mice with IL-18BP reduces inflammation in the animal.

From the experiments outlined above, the following conclusions can be drawn:

Administration of IL-18BP decreases the clinical severity of arthritis

IL-18BP inhibits further progression or spreading of the disease

Administration of IL-18BP decreases oedema

Administration of IL-18BP decreases cartilage destruction

Serum IL-6 levels are diminished and IgG1/IgG2a anti-CII ratios decreased after IL-18BP therapy.

The data presented above indicate that neutralisation of IL-18 bioactivity after disease onset represents a disease-modifying anti-rheumatic therapy. The results clearly demonstrate that IL18 blockade reduces the clinical progression of arthritis and more importantly stops progression of cartilage and bone destruction. IL18 blockade by IL-18BP, anti-IL-18 antibodies or by any other IL-18 blocking agent therefore represents a new disease-modifying anti-rheumatic therapy.

The foregoing description of the specific embodiments reveal the general nature of the invention so that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation.

Part III: Examples 11 and 12 relating to Inflammatory Bowel Disease

### Example 11

### IL-18BP Expression by Endothelial Cells and Macrophages During Active Crohn's Disease

Collection of specimens

Intestinal mucosal biopsies were isolated from surgical resection specimens from patients with CD or UC. Fourteen CD patients (three males and eleven females) with a mean age of 37.8 years (range 20-78 years) and a disease duration of 8.3 years (range 1-21 years) were included. In eight patients, disease was located in the ileum and in six in the colon. Twelve patients were on immunosuppressive drugs. The diagnosis of active CD was made by histo-pathological examination and based on the following criteria: presence ulcerations, increased number of inflammatory cells and transmural inflammation. Seven patients with active CD and seven patients with non-active disease were identified. No significant differences in age, disease localisation, sex, medication and disease duration were observed between active and non-active CD patients. The mean age of the 5 UC patients (three males and two females) was 37.6 years (range 30-44 years). In all patients disease was located in the colon and all were on immunosuppressive therapy. Average disease duration was 4 years (range 1-9 years). Control samples were obtained from 5 patients undergoing a resection for non-IBD related disorders (three males and two females). Mean age of this group was 55.2 years (range 24-76 years). In all patients, disease was located in the colon.

Semiquantitative RT-PCR for human IL-18 and IL-18bp

Total RNA was extracted from frozen intestinal biopsies of patients with CD, with UC or of control patient. RNA extraction was performed using Trizol (Gibco) according to the manufacturer's instructions. The samples obtained, were quantitated by measuring the absorbance at 260 nm. RNA integrity was assessed by electrophoresis on 1% agarose gels. cDNA was synthesized from 1 μg of total RNA using the Promega reverse transcription system according to the manufacturer's protocol. PCR reactions were performed in a total volume of 50 μl in presence of 1U of AmpliTaq DNA Polymerase (Perkin Elmer, Roche, U.S.A), 2.5 mM dNTPs (Amersham, U.S.A), and 50 pmoles of forward and reverse PCR primers. Reactions were incubated in a PTC-200 Peltier Effect Thermal Cycler (MJ Research, U.S.A) under the following conditions: denaturation 1 min at 94° C., annealing for 1 min at 55° C. and extension for 1 min at 72° C. The optimum number of cycles for IL-18BP, IL-18 and β-actin before saturation of the bands was determined (31, 28 and 25, respectively). PCR primers were designed based on the published sequences (AF110799, D49950, X00351) as follows: IL-18, reverse 5′-GCGTCACTACACTCAGCTAA-3′; forward 5′-GCCTAGAGGTATGGCTGTAA-3′; IL-18BP, forward 5′-ACCTGTCTACCTGGAGTGAA-3′; reverse 5′-GCACGAAGATAGGAAGTCTG-3′; β-actin, reverse 5′-GGAGGAGCAATGATCTTGATCTTC-3′; forward 5′-GCTCACCATGGATGATGATATCGC-3′. To exclude the amplification of genomic DNA contaminating the samples, PCR reactions were performed in the absence of the cDNA template. PCR products (10 μl) were analysed on 1% agarose gels electrophoresed in 1×TAE buffer. The size of PCR products was verified by comparison with a 1 Kb ladder (Gibco) following staining of the gels. Relative quantification of ethidium-bromide stained bands was performed under UV light using the Kodak Digital Sciences analytical software, and was reported as the ratio of target gene (hIL-18BP, hIL-18) to the housekeeping gene (hβ-actin).

Generation of monoclonal antibodies directed against hIL-18BP.

BALB/c mice were injected subcutaneously into the four limbs as well as intranuckally, with 50 μg of isoform a rhIL-18BP-6his (purified from chinese hamster ovary cells, Interpharm Laboratories, Nes Ziona, Israel) in PBS with adjuvant (MPL+TDM Emulsion, RIBI Immunochem Research, Inc.) on days 0, 7 and 28. Four days after the 3^{rd} immunisation, the lymph nodes were obtained and digested with 2.4 ?g/ml collagenase (collagenase IV, Worthington Biochemical Corp.) and 0.1% Dnase (Sigma). Isolated cells were then fused with the Sp2/0 myeloma cells using PEG 1000 (FLUKA). The cells were resuspended in DMEM-F12, 10% FCS (Gibco) in the presence of HAT (hypoxanthine, aminopterin, thymidine) and distributed in 96 well plates at a concentration of 5×10⁻⁴ cells/ml. Hybridoma culture supernatant samples were screened for the presence of reactive antibodies in a direct screening assay. For this, ELISA plates were coated with goat anti-mouse F(ab′)₂ antibodies (Jackson Immuno Research, Milan analytica, Switzerland), hybridoma culture supernatants were added followed by biotinylated rhIL-18BP-6his (purified from COS cells as described (Novick, et al. 1999), with or without rhIL-18 (purified from recombinant E. Coli, Serono Pharmaceutical Research Institute, Geneva), and finally streptavidin-horseradish peroxidase (HRP) (Jackson Immuno Research, Milan analytica, Switzerland) developed using o-phenylenediamine (OPD) (Sigma). Non-neutralising antibodies were selected and subcloned. In this study, 95-H20, a mouse IgG1 monoclonal antibody, was used.

Immunohistochemical studies for the localisation of IL-18bp-positive cells

Tissue specimens were snap frozen and stored at −80° C. Serial cryosections (10 μm) were obtained, mounted on poly-L-lysine-coated Superfrost/Plus glass slides (Polylabo, Plan-les-Ouates, Switzerland) and fixed in ice-cold acetone. Localisation of the human IL-18BP protein was analysed by immunohistochemistry using Mab 95-H20. After a brief rehydration in PBS, sections were pre-incubated for 30 minutes in PBS supplemented with 2% Fetal Calf Serum (FCS) (Cansera, Ontario, Canada), 1% human serum (AB⁺ serum, Transfusion Center, Annemasse, France) and 0.5% Bovine Serum Albumin (BSA) (Sigma, St. Louis, Mo., U.S.A). Endogenous peroxidase activity was blocked by placing the slides in a solution of PBS, 2% FCS, 1% human serum, 0.5% BSA and 1% hydrogen peroxide (H₂O₂) (Fluka, Switzerland) for 1 h. Following a rinse in PBS, sections were incubated overnight with non-diluted culture supernatant of the Mab 95-H20. Following another wash in PBS, sections were incubated with biotinylated goat anti-mouse antibody (Jackson Immuno Research, Milan analytica, Switzerland) (5 ?g/ml) in PBS containing 0.5% BSA for 1 h. The sensitivity of the staining was increased by incubating with an avidinDH/biotinylated HRP complex (Vectastain Elite ABC Kit, Vector Laboratories, CA, U.S.A) for 30 min. Slides were then washed with PBS and developed using a solution of 30% H₂O₂, 3,3-amino-9-ethyl carbazole (AEC) (Sigma), N,N-dimethylformamide (Merck) in acetate buffer, pH 5. Following counter-staining with hematoxylin (Sigma), sections were coated with glycerol and cover slips applied. A mouse IgG1 antibody (R and D system) was used as the isotype control.

In order to identify the cellular localisation of human IL-18BP, two colours immunohistochemical study was performed on mucosal intestinal sections. After a 10 min. rehydration in PBS, sections were pre-incubated for 30 minutes in PBS supplemented with 2% FCS, 1% human serum and 0.5% BSA. For colocalization with endothelial cells, sections were incubated overnight with biotinylated Mab 95-H20 (20 μg/ml) mixed with FITC-conjugated mouse anti-human CD31 (1:50) (Pharmingen, CA, U.S.A) in PBS/0.5% BSA. For colocalization with macrophages, the sections were incubated overnight with biotinylated Mab 95-H20 (20 μg/ml) mixed with FITC-conjugated anti-human CD68 (1:25) (Dako, Denmark). Following a wash in PBS, streptavidin Texas-Red (Southern Biotechnology Associates, AL, U.S.A) was added for 1 h. Slides were again washed with and sections were coated with moviol and cover slips were applied. The biotinylated mouse IgG1 antibody (Pharmingen) followed by streptavidin Texas-Red was used as isotype control.

Cell Culture

Human umbilical vein endothelial cells (HUVECs) (Clonetics Corp., San Diego, Calif.) were cultured using Endothelial Cell Growth Medium (EGM) supplemented with human recombinant epidermal growth factor (hEGF) (10 ng/ml), hydrocortisone (1 ?g/ml), gentamicin and amphotericin B (50 ??g/ml), bovine brain extract (BBE) (3 mg/ml) and 2% fetal bovine Serum (FBS) (Clonetics Corp., San Diego, Calif.) according to the manufacturer's instructions. Tissue culture dishes were pre-coated with human fibronectin (10 ?g/cm²) (Boehringer, Mannheim). Cells were incubated in a humidified 5% CO₂ incubator and experiments were performed using the HUVECs at passage 3. HUVECs were treated with human IL-1β (10 ng/ml), TNFα (10 ng/ml) and IFNγ (20 ng/ml) (R and D system, Germany) for 24 h. At the end of the culture period, cells were collected, RNA isolated and subjected to RT-PCR for IL-18BP and IL-18 mRNA transcript analysis. Supernatants were collected and analysed for IL-18BP and IL-18 protein expression by ELISA.

The human monocytic cell line THP-1 was maintained in a suspension culture in RPMI medium supplemented with 10% heat inactivated FCS, L-glutamine (2 mM), penicillin-streptomycin (10 U/ml) (Gibco BRL, Life Technologies) and β-mercaptoethanol (50 ?M) (Fluka). They were incubated in a humidified 5% CO₂ incubator and passaged at 1:10 every 5 days. Three days before experimentation, human monocytic cells were differentiated at a density of 0.4×10⁶ cells/ml with Vitamin D3 (80 nM) (Biomol Research Laboratories, USA) and left to adhere. Once differentiated, LPS (100 ng/ml) (Calbiochem), human IL-1β (10 ng/ml), TNFα (10 ng/ml) and IFNγ (20 ng/ml) were added to the cell cultures. At 48 h, supernatants were collected and analyzed for IL-18BP and IL-18 protein expression by ELISA.

Measurement of human IL-18bp and IL-18 production

The presence of IL-18BP was evaluated by ELISA in the cell-free supernatants from HUVECs, non-stimulated and stimulated for 24 h with a cocktail of cytokines (IL-1β, TNFα, IFNγ), as well as from the THP-1 cell line, non-stimulated and stimulated for 48 h (LPS, IL-1β, TNFα. IFNγ). For this, plates were coated overnight with a capture Mab (clone 657.27 at 0.5 μg/100 ?l/well, Interpharm Laboratories, Nes Ziona, Israel) directed against rhIL-18BP (isoform a). Soluble hIL-18BP was then detected using a rabbit polyclonal antibody (diluted at 1/10,000) raised against rhIL-18BP-6his (purified from chinese hamster ovary cells, Interpharm Laboratories, Nes Ziona, Israel), followed by incubation with a peroxidase conjugated affinity purified goat anti-rabbit IgG (diluted at 1/20,000) (Jackson Immuno Research, Milan analytica, Switzerland). The capture Mab as well as the rabbit polyclonal antibody were tested by Western Blot in order to confirm IL-18BP specificity. Recombinant hIL-18BP-6his was used as standard. The sensitivity of the ELISA was 100 pg/ml. In paralell, levels of IL-18 was quantitated using the human IL-18 ELISA Kit (MBL, Immunotech). The sensitivity of the ELISA was 12.5 pg/ml.

Expression of hIL-18BPa-His6 in CHO cells.

Recombinant human IL-18BP (hIL-18BPa His6-tag) was purified from Chinese hamster ovary cells (Interpharm Laboratories, Nes Ziona, Israel).

### Results

Expression of IL-18BP mRNA transcripts in intestinal biopsies

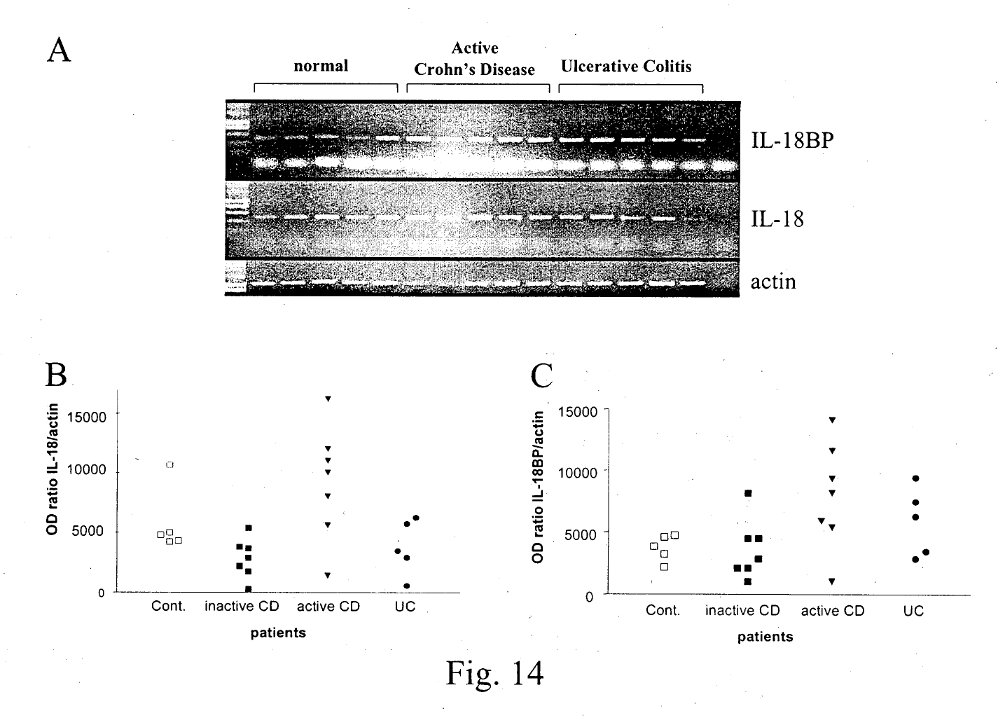

Analysis for IL-18BP mRNA expression was performed by RT-PCR on tissue homogenates from colonic surgical specimens from patients with either active CD, non active CD, or UC and from non-inflamed intestinal tissues (FIG. 14). IL-18BP and actin transcripts were detected in all intestinal homogenates tested. Similarly, transcripts for IL-18 were found in all tissue homogenates either from CD, UC or non-IBD controls (FIG. 14 A). The ratio of IL-18BP or IL-18 to control actin mRNA levels demonstrated a statistically significant increase (as described below) in the amount of transcripts for both IL-18BP and IL-18 in biopsies obtained from patients with active CD in comparison to biopsies of patients with non active CD, UC and non IBD controls (FIGS. 14 B and C). These data show that IL-18BP is up-regulated in mucosal tissue during active CD and provide the first evidence that the level of IL-18BP expression clearly differentiates active CD from non-active CD, UC and non-IBD controls.

Statistical analysis on the expression of IL-18BP mRNA transcripts in intestinal biopsies

An analysis of variance was carried out, pooling all the available data together. The output clearly indicated a statistical outlier regarding the results for one of the patients from the Active CD group (very high OD ratio for IL 18, namely 16252, and very low OD ratio for IL 18BP, namely 1058.). This single and very atypical couple of measurements did not allow validation of the ANOVA model (p-value of the test of Shapiro-Wilks for the normality of the residuals<0.0001). Thus it was decided to ignore this couple of measurements in the statistical analysis.

The ANOVA model used took into account the factor Group (Control, Active CD, Inactive CD, and UC), Protein (IL-18 or IL-18BP), and Patient number (23 patients). There is a significant difference among group (p-value<0.0001). It is also interesting to note that the difference of OD ratio between IL-18 and IL-18BP is not significant (p-value=0.369). Moreover, the correlation between IL-18 and IL-18BP expression was also performed. The coefficient of correlation between IL-18 and IL-18BP is equal to 0.67, which suggests a strong link between the OD ratio measured for IL-18 and IL 18-BP. Following up the results regarding the Group effect, the method of Scheffé was used to compare the different groups. It can be concluded that the OD ratio for Active CD is significantly greater than for the Control (+3280), the UC (+2590), and especially the Inactive CD (+4580) both for IL-18BP and IL-18 expression.

Immunohistochemical localization of IL-18BP in intestinal tissues.

To assess the cellular expression of IL-18BP in situ, immunohistochemistry using anti-hIL-18BP specific monoclonal antibodies was performed on cryosections prepared from intestinal tissues obtained from patients with active CD and from non-IBD controls. IL-18BP positive cells were detected in the lamina propria, the submucosa and within the muscle layer (not shown). Positively stained mononuclear cells present within the lamina propria and the submucosa possessed abundant cytoplasm, vesicular retiform nuclei, and were morphologically consistent with tissue macrophages. In the muscle layer, positively stained cells had abundant cytoplasm, with some time open lumen in the middle, suggesting positive staining of microvessels, and were morphologically consistent with endothelial cells. Large vessels were also specifically stained by anti-hIL-18BP monoclonal antibodies. The significant increase in positively stained cells observed in specimens obtained from patients with active CD when compared with specimens obtained from non-IBD controls correlated with the increased IL-18BP expression observed by RT-PCR analysis. Adjacent sections were incubated with the corresponding mouse isotype control.

Identification of il-18bp producing cells presents in mucosal biopsies

IL-18BP positive cells present in inflamed intestinal tissues were identified using specific markers of macrophages (anti-CD68), and endothelial cells (anti-CD31) (not shown). CD68 positive cells (in green) and IL-18BP positive cells (in red) were detected within the lamina propria and the submucosa of intestinal tissues from active CD (not shown). CD31 positive cells (in green) and IL-18BP positive cells (in red) were detected in the submucosa. To confirm that macrophages and endothelial cells were also positive for IL-18BP, both colours, the green from anti-CD68 or anti-CD31 and the red from anti-IL-18BP were analysed together demonstrating that all the cells that bound anti-IL-18BP antibodies were either CD68 positive or CD31 positive (orange-yellow colour). The double immunolabeling demonstrated that macrophages and endothelial cells were the major source of IL-18BP staining within inflamed tissues obtained from patients with CD.

Expression of Il-18bp mRNA and protein by endothelial cells

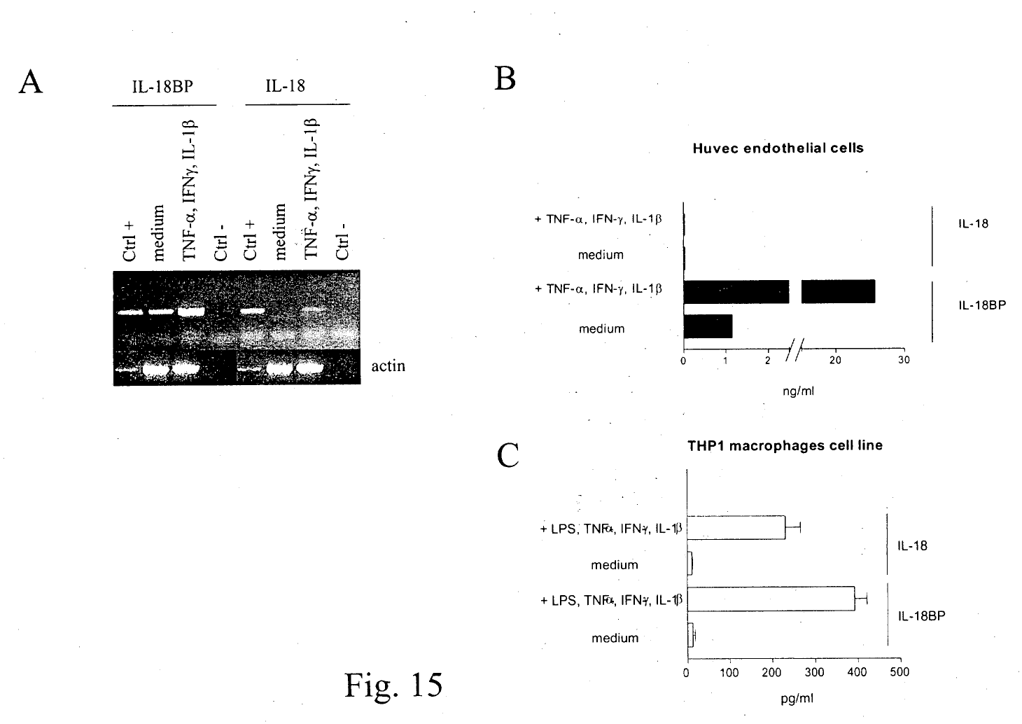

To investigate the capability of human endothelial cells to produce IL-18BP as well as to confirm the result found by immunostaining and RT-PCR on total biopsies, further RT-PCR experiments were performed on human umbilical vein endothelial cells (HUVECs) (FIG. 15 A). Endothelial cells were treated with a cocktail of cytokines (hIL-1β, hTNFα, hIFNγ) and collected after 24 h for RNA extraction and PCR analysis. The ratio of IL-18BP to control actin mRNA levels demonstrated an increase in the amount of IL-18BP transcripts in treated cells compared with non stimulated cells after 24 h. Moreover, IL-18BP mRNA seemed to be constitutively expressed in endothelial cells. The IL-18 mRNA level was also analyzed and demonstrated a slight increase in treated cells. However IL-18 mRNA is not expressed in non-stimulated endothelial cells.

ELISA with culture supernatants of non-stimulated cells (medium) and HUVECs treated for 24 h revealed that IL-18BP protein was present in both the medium and the stimulated cells with a 30× increase after 24 h of stimulation (FIG. 15 B).

Expression of IL-18bp protein by a monocytic cell line (THP-1)

The expression of IL-18 and IL-18BP was analyzed in culture supernatants of non-stimulated and stimulated, differentiated THP-1 cells by ELISA (FIG. 15 C). This experiment revealed an increase of IL-18BP expression after 48 h of stimulation with LPS, hIL-1β, hTNFα, hIFNγ. In parallel, IL-18 secretion was increased after stimulation (FIG. 15 C).

### Summary

In the present study, the expression of IL-18BP and localisation in mucosal tissue obtained from patients with Crohn's Disease and Ulcerative Colitis was characterised. Using a semiquantitative RT-PCR protocol, IL-18BP mRNA transcripts were found to be increased in mucosal biopsies of patients with active Crohn's Disease compared with Ulcerative Colitis and noninflamed control patients. Immunohistochemical analysis of mucosal biopsies localised IL-18BP protein within endothelial cells and in macrophages that infiltrate the mucosa during Crohn's Disease. The IL-18BP expression by endothelial cells and activated macrophages was confirmed in the primary human umbilical vein endothelial cells (HUVECs) and in the THP1 monocytic cell line, stimulated in vitro. Following stimulation, these cells secreted bioactive IL-18BP.

### Example 12

### Treatment with IL-18 Inhibitors Ameliorates Experimental Colitis

### Material and Methods

Mice and induction of colitis

The Animal Studies Ethics Committee of the University of Amsterdam, The Netherlands, approved all experiments. BALB/c mice were obtained from Harlan Sprague Dawley Inc (Horst, the Netherlands). The mice were housed under standard conditions, and supplied with drinking water and food (AM-II 10 mm, Hope Farms, Woerden, The Netherlands).

Experiments were conducted in 8 and 10 weeks old female BALB/c mice. Colitis was induced by rectal administration of two doses (separated by a 7 day interval) of 2 mg 2,4,6-trinitrobenzene sulfonic acid (TNBS) (Sigma Chemical Co, St Louis, Mo., USA) in 40% ethanol (Merck, Darmstadt, Germany), using a vinyl catheter that was positioned 3 centimetres from the anus (10 mice per group). During the instillation, the mice were anaesthetized using isoflurane (1-chloro-2,2,2,-trifluoroethyl-isoflurane-difluoromethyl-ether, Abbott Laboratories Ltd., Queenborough, Kent, UK), and after the instillation they were kept vertically for 60 seconds. Control mice underwent identical procedures, but were instilled with physiological salt. All mice were sacrificed at 9 days following the first TNBS administration (i.e. 48 hours following the second TNBS challenge).

Mice were treated with human IL-18BP in 500 μl 0.9% saline intra-peritoneally.

HIL-18BP is a 6 times histidine tagged human recombinant protein produced in a CHO expression system. The hIL-18BP was biological active as it inhibited IFNγ production in the KG-1 cell line and reduces IFNγ production by mouse spleen cells (Kim et al., 2000).

Assessment of inflammation

Body weights were recorded daily. Spleen, caudal lymph nodes and colons were harvested upon sacrifice. The colons were removed through a midline incision and opened longitudinally. After removal of faecal material, the wet weight of the distal 6 cm was recorded and used as an index of disease-related intestinal wall thickening. Subsequently, the colons were longitudinally divided in two parts, one of which was used for histological assessment.

Histological analysis

The longitudinally divided colons were rolled up, fixed in 4% formaline and embedded in paraffin for routine histology. Two investigators who were blinded for the treatment allocation of the mice scored the following parameters: 1) percentage of area involved, 2) hyperplasia of follicle aggregates, 3) oedema, 4) erosion/ulceration, 5) crypt loss and 6) infiltration of mono- and polymorphonuclear cells. The percentage of area involved and crypt loss was scored on a scale ranging from 0 to 4 as follows: 0, normal; 1, less than 10%; 2, 10%; 3, 10 to 50%; 4, more than 50%. Erosions were defined as 0 if the epithelium was intact, 1 for the involvement of the lamina propria, 2 ulceration's involving the submucosa, and 3 when ulcerations were transmural. The severity of the other parameters was scored on a scale 0 to 3 as follows: 0, absent; 1, weak; 2, moderate; 3, severe. This score ranges from 0 to a maximum of 26 points.

Colon homogenates

Colon was harvested and homogenates were made with a tissue homogenizer in 9 volumes of Greenburger lysis buffer (300 mM NaCl, 15 mM Tris, 2 mM MgCl, 2 mM Triton (X-100), Pepstatin A, Leupeptin, Aprotinine (all 20 ng/ml), pH 7.4) Tissue was lysed for 30 minutes on ice followed by two times centrifugation (10 min., 14.000 g). Homogenates were stored on −20° C. until use.

Cell culture and ELISA for cytokines

For preparing spleen and caudal lymph node cell suspension, filter cell strainers (Becton/Dickinson Labware, New Jersey, USA) were used. Cells were suspended in RPMI 1640 medium (BioWhittaker-Boehringer, Verviers, Belgium) containing 10% FCS and ciproxin (10 μg/ml) (Sigma Chemical Co., St. Louis, Mo., USA). Spleen cells were centrifuged with sterile Ficoll (Pharmacia, Uppsala, Sweden), mononuclear cells were transferred to RPMI and the cell supsensions were counted. A total number of 1×10⁵ cells per mice was incubated in 200 μl RPMI (BioWittaker Europe, A Cambrex Company, Verviers, Belgium) containing antibiotics and 10% fetal calf serum in triplicate wells. Cells were stimulated by precoating with anti-CD3 antibody (1:30 concentration; 145.2C11 clone) and soluble anti-CD28 antibody (1:1000 concentration; Pharmingen). Supernatants were removed after 48 hours and IFN-γ (Pharmingen) and TNF-α (R&D systems, Abingdon, United Kingdom) concentrations measured by ELISA assay.

Flow cytometry

Isolated spleen cells were washed with Facs buffer (PBS, containing 0.5% BSA, 0.3 mmol/L EDTA and 0.01% sodium azide) and were kept on ice for the remainder of the procedure. 2.10⁵ cells per well (96 well v-shape microplate, Greiner B.V. labor techniek, Alphen aan de Rijn, The Netherlands) were incubated with the following antibodies (mAbs): Cy-chrome-conjugated rat anti-mouse CD4 (clone RM4-5), Fitc-conjugated rat anti-mouse CD69 and Fitc-conjugated rat anti-mouse CD25 (Pharmingen, San Diego, Calif.). Lymphocytes were gated by forward and side scatter using a FACScan flow cytometer in conjunction with the Facscan software (Becton Dickinson, Mountain View, USA) and 5000 cells were counted. Results are expressed as the percentage of gated cells positive for the mAbs used.

Statistical analysis

Values are given as mean and SEM per treatment group. Differences between groups were analysed using the non-parametric Mann-Withney U test. Weight changes in time were analysed by one-way analysis of variance. P<0.05 was considered significant. SPSS statistical software (SPSS inc., Chicago, USA) was used for all analyses.

### Results

IL-18BP protects against weight loss in a murine model of colitis

To investigate the role of IL-18 in experimental colitis and in particular the protective effect of IL-18 binding protein (IL-18BP), TNBS colitis was induced in BALB/c mice. This model consists of Local exposure to tri-nitrobenzene sulfonic acid (TNBS) in 40% ethanol. It evokes delayed type hypersensitivity reaction to the hapten (trinitrophenyl) modified self Antigen, and the response is a Th1-type with enhanced proinflammatory cytokine production

Mice were treated with human IL-18BP or control intra-peritoneally (ip) on a daily base.

Daily intraperitoneal doses ranging from 12.5 μg to 50 μg hIL-18BP did not affect disease severity (data not shown). However, using a dose of 200 μg hIL-18BP daily administered intraperitoneally was effective in reducing the weight loss in connection with the induction of the disease.

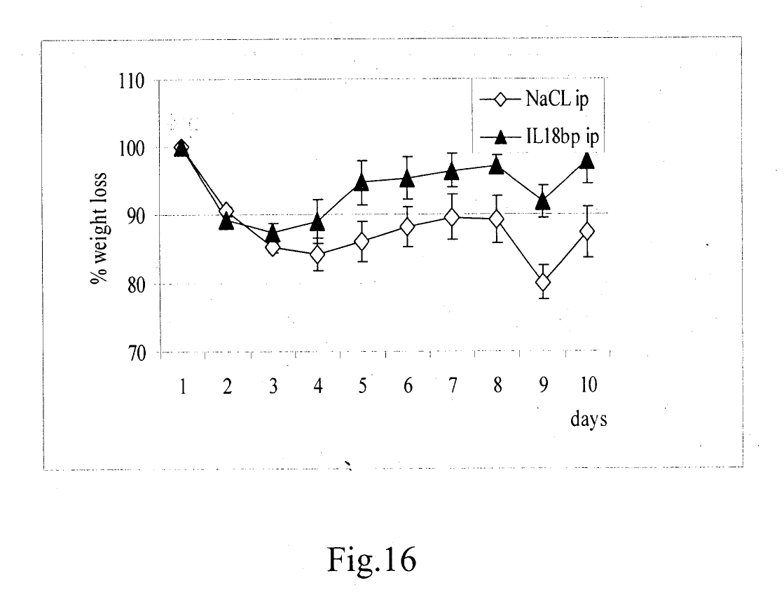

As expected, intrarectal instillation of TNBS resulted in diarrhoea and wasting. As shown in FIG. 16, animals in both treatment groups losing 15% of the baseline weight at day 3. However, in contrast to control mice, starting at day 4 after TNBS instillation, hIL-18BP-treated animals rapidly recovered from the initial weight loss, returning to baseline body weight at day 8. In control mice, the second administration of TNBS on day 8 again resulted in a significant weight loss, which was essentially prevented by hIL-18BP administration. Administration of hIL-18BP in saline-treated mice had no effect (data not shown). Hence, administration of hIL-18BP significantly attenuated weight loss associated with TNBS-induced colitis (p<0.05).

Effects on inflammatory parameters

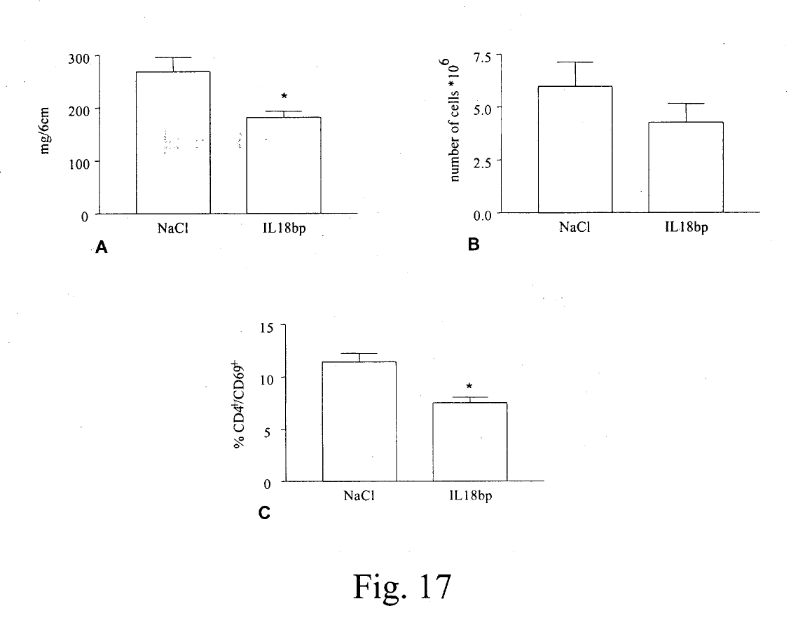

On day 10 mice were sacrificed and the weight of the last 6 centimetres of the colon was determined (FIG. 17 A). In TNBS colitis the colon weight increased as compared to saline treated mice. This increase in weight was significantly less in hIL-18BP-treated mice (181.6 mg±11.4 as compared to 268 mg±27.3 in saline treated mice (p<0.05)). It has been previously reported that TNBS colitis is associated with increased cell migration into caudal lymph nodes (Camoglio et al., 2000). IL-18BP treatment reduced the number of cells invading the caudal lymph node compared to the number of cells in the caudal lymph node of TNBS mice treated with saline (FIG. 17 B).

A change in CD69 expression, which is an early T lymphocyte activation marker, was determined by Facscan analysis (FIG. 17 C). The percentage of CD4⁺ spleen cells expressing CD69 was 11.4% in TNBS-treated mice, but in TNBS mice treated with hIL-18BP the percentage of CD4⁺/CD69⁺ was 7.3% (P<0.05).

Cytokine production of spleen, caudal lymph node cells and colon homogenates

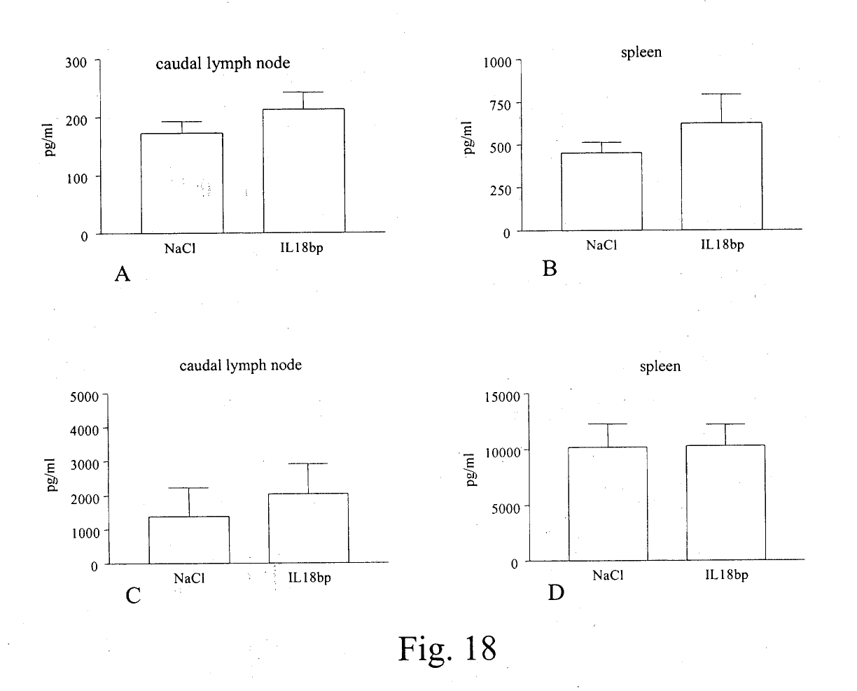

To investigate the effect of hIL-18BP on the ability of T-lymphocytes to produce pro-inflammatory cytokine synthesis following activation of the T-cell receptor, cells were isolated from caudal lymph node and spleen and stimulated these for 48 hours with anti CD3/CD28 antibodies. In the supernatants IFNγ and TNFα production was measured (FIG. 18). No significant differences were observed between cytokine production of hIL-18BP mice and control treated mice. Hence, neutralisation of IL-18 by hIL-18BP did not cause a generalised reduction of the ability of T-lymphocytes to respond to T-cell receptor activation.

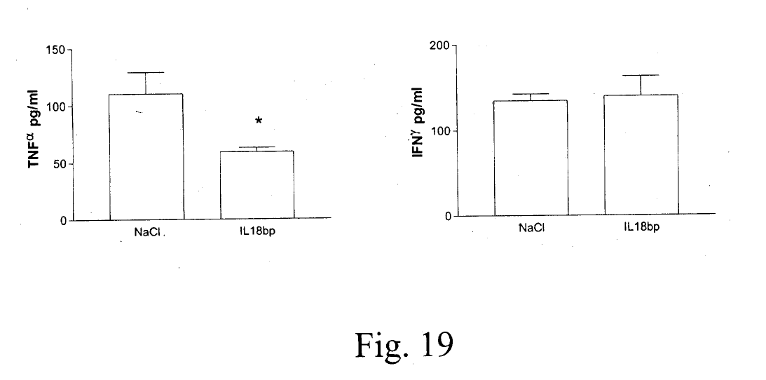

Colon homogenates were analysed for their cytokine levels, thereby measuring the local production of cytokines (FIG. 19). No difference in IFNγ levels were detected in colon homogenates of TNBS mice and TNBS mice treated with hIL-18BP (134 pg/ml±7.8 and 139 pg/ml±23 respectively). However, TNFα levels were significantly reduced in colon homogenates of mice treated with hIL-18BP from110 pg/ml±3 in TNBS mice to 59 pg/ml+2.7 in hIL-18BP treated TNBS mice.

Histological findings

To investigate if the hIL-18BP-mediated reduction of inflammatory parameters also affected the histological score, histopathology was performed on paraffin-sections. The total inflammatory score on histology was significantly decreased in the hiL-18BP treated mice compared to control treated mice (15.9±1.1 in non-treated mice to 9.8±1.3 in hIL-18BP treated mice) (not shown), mainly as a result of a reduction of the number of leukocytes infiltrating the mucosa (p<0.05). A remarkable finding was the complete absence of mucosal ulcerations in IL-18BP-treated mice (p<0.05). The findings are summarized in Table 2 below. The complete prevention of ulcerations in IL-18BP treated mice is particularly remarkable.

TABLE 2
Different items of the colitis core of TNBS
mice treated with saline or rhIL-18BPa.

| | | hIL-18BP treated |
|---|---|---|
| | Control treated TNBS mice | TNBS mice |
| % Area involved | 3.4 ± 0.4 | 3.2 ± 0.5 |
| Follicle aggregates | 2.0 ± 0.4 | 1.5 ± 0.4 |
| Oedema | 2.1 ± 0.3 | 1.3 ± 0.3 |
| Fibrosis | 0.85 ± 0.26 | 0.50 ± 0.2 |
| Ulcerations | 2.0 ± 0.3 | 0.0 ± 0.0* |
| Crypt loss | 1.7 ± 0.3 | 1.0 ± 0.3 |
| Polymorph nuclear cells | 2.6 ± 0.2 | 1.3 ± 0.2* |
| Mono nuclear cells | 1.1 ± 0.1 | 0.33 ± 0.21* |

Data are presented as mean ± SEM on a scale from 0-4,
*represents a signicant difference.

Anti-mIL-18 polyclonal antibodies protect from disease in a mouse model of dextran sulfate sodium-induced colitis

In this model, dextran sulfate sodium (DSS), id was given within the drinking water starting at Day 0 until the sacrifice of the animals. The anti-IL-18BP polyclonal antibodies were administrated at day 0, 4 and 8, i.p. The doses were 200 and 400 ?l of rabbit serum. The highest dose (400 μl) gave a significant reduction in weight loss, clinical score, rectal bleeding and colon shortening (not shown). The rabbit anti-mIL-18 treatment showed a delay in the initiation of the disease and prevented the progression (not shown).

### Summary

Example 12 presented above demonstrate that neutralisation of IL-18 by administration of either hIL-18BP or polyclonal antiserum against IL-18 efficiently reduces the severity of experimentally induced colitis in mice.

Mice treated daily with hIL-18BP intra-peritoneally rapidly recovered from a primary weight loss as compared to control treated mice. Other parameters of colonic inflammation measured by colon weight and influx of cells in the caudal lymph node were reduced in hIL-18BP treated mice. The histopathology of the treated mice was characterised by a reduction of severity of the tissue destruction (ulcerations) and the amount of infiltrating cells was considerably reduced. The effect of hIL-18BP was also systemic, as demonstrated by reduced expression of CD69 by spleen cells.

The local production of TNFα, measured in colon homogenates, was significantly reduced in TNBS mice treated with hIL-18BP. This indicates that TNFα plays an important role in disease progression. IFNγ levels were comparable between TNBS mice and TNBS mice treated with hIL-18BP, which may be explained by the redundancy of IFNγ-inducing stimuli.

In conclusion, the data presented above demonstrate the beneficial effect of inhibitors of IL-18 in the treatment of inflammatory bowel diseases.

### REFERENCES

1. Afford, S. C., et al., Distinct patterns of chemokine expression are associated with leukocyte recruitment in alcoholic hepatitis and alcoholic cirrhosis. J Pathol, 1998. 186(1): p. 82-9.

2. Anderson, D. M., et al., A homologue of the TNF receptor and its ligand enhance T-cell growth and dendritic-cell function. Nature, 1997. 390(6656): p. 175-179.

3. Baroni, G. S., et al., Hepatic stellate cell activation and liver fibrosis are associated with necroinflammatory injury and Th1-like response in chronic hepatitis C. Liver, 1999. 19(3): p. 212-9.

4. Bird, G. L., et al., Increased plasma tumor necrosis factor in severe alcoholic hepatitis. Ann Intern Med, 1990. 112(12): p. 917-20.

5. Bollon, D. P., et al. (1980) J. Clin. Hematol. Oncol. 10:39-48.

6. Botstein, D., et al. (1982) Miami Wint. Symp. 19:265-274.

7. Broach, J. R., in “The Molecular Biology of the Yeast Saccharomyces: Life Cycle and Inheritance”, Cold Spring Harbor Laboratory, Cold Spring Harbor, N.Y., pp. 445-470 (1981).

8. Broach, J. R., (1982) Cell 28:203-204.

9. Bym R. A. et al., 1990, Nature (London) 344:667-670.

10. Camoglio L, te Velde A A, de Boer A, ten Kate F J, Kopf M, van Deventer S J. Hapten-induced colitis associated with maintained Th1 and inflammatory responses in IFN-gamma receptor-deficient mice. Eur J Immunol 2000;30:1486-95.

11. Car, B. D., V. M. Eng, B. Schnyder, L. Ozmen, S. Huang, P. Gallay, D. Heumann, M. Aguet, and B. Ryffel. 1994. Interferon gamma receptor deficient mice are resistant to endotoxic shock. J. Exp. Med. 179:1437-44 issn: 0022-1007.

12. Chater, K. F. et al., in “Sixth International Symposium on Actinomycetales Biology”, Akademiai Kaido, Budapest, Hungary (1986), pp. 45-54).

13. Conti, B., J. W. Jahng, C. Tinti, J. H. Son, and T. H. Joh. 1997. Induction of interferon-gamma inducing factor in the adrenal cortex. J. Biol. Chem. 272:2035-2037.

14. Dao, T., K. Ohashi, T. Kayano, M. Kurimoto, and H. Okamura. 1996. Interferon-gamma-inducing factor, a novel cytokine, enhances Fas ligand-mediated cytotoxicity of murine T helper 1 cells. Cell-Immunol. 173:230-5 issn: 0008-8749.

15. Dayer, J-M (1999). J. Cin. Inv. 104, 1337-1339.

16. Desreumaux P, Brandt E, Gambiez L, Emilie D, Geboes K, Klein O, Ectors N, Cortot A, Capron M, Colombel J F. Distinct cytokine patterns in early and chronic ileal lesions of Crohn's disease. Gastroenterology 1997;113:118-126.

17. DiDonato, J A, Hayakawa, M, Rothwarf, D M, Zandi, E and Karin, M. (1997), Nature 388, 16514-16517.

18. Elliott, M. J., Maini, R. N., Feldmann, M., Long-Fox, A., Charles, P., Bijl, H., and Woody, J. N., 1994, Lancet 344, 1125-1127.

19. Engelmann, H., D. Aderka, M. Rubinstein, D. Rotman, and D. Wallach. 1989. A tumor necrosis factor-binding protein purified to homogeneity from human urine protects cells from tumor necrosis factor toxicity. J. Biol. Chem. 264:11974-11980.

20. Engelmann, H., D. Novick, and D. Wallach. 1990. Two tumor necrosis factor-binding proteins purified from human urine. Evidence for immunological cross-reactivity with cell surface tumor necrosis factor receptors. J. Biol. Chem. 265:1531-1536.

21. Fantuzzi, G., et al., IL-18 regulation of IFN-g production and cell proliferation as revealed in interleukin-1b converting enzyme-deficient mice. Blood, 1998. 91: p. 2118-2125.

22. Fiore, G., et al., Liver tissue expression of CD80 and CD95 antigens in chronic hepatitis C: relationship with biological and histological disease activities. Microbios, 1999. 97(386): p. 29-38

23. Galle, P. R., et al., Involvement of the CD95 (APO-1/Fas) receptor and ligand in liver damage. J Exp Med, 1995. 182(5): p. 1223-30.

24. Gracie, A J, Forsey, R J, Chan, W L, Gilmour, A, Leung, B P, Greer, A R, Kennedy, K, Carter, R, Wei, X-Q, Xu, D., Field, M, Foulis, A, Liew, F Y, and McInnes, I B. (1999). J. Clin. Inv. 104: 1393-1401

25. Grantham (1974), Science, 185. 862-864.

26. Grove, J., et al., Association of a tumor necrosis factor promoter polymorphism with susceptibility to alcoholic steatohepatitis [see comments]. Hepatology, 1997. 26(1): p. 143-6.

27. Gryczan, T., “The Molecular Biology of the Bacilli”, Academic Press, NY (1982), pp. 307-329).

28. Gutkind, J. S., et al., A novel c-fgr exon utilized in Epstein-Barr virus-infected B lymphcytes but not in mormal monocytes. Molec. Cell. Biol., 1991. 11: p. 1500-1507.

29. Harada, K., et al., In situ nucleic acid hybridization of cytokines in primary biliary cirrhosis: predominance of the Th1 subset. Hepatology, 1997. 25(4): p. 791-6.

30. Heremans, H., J. Van Damme, C. Dillen, R. Dijkmans, and A. Billiau. 1990. Interferon gamma, a mediator of lethal lipopolysaccharide-induced Shwartzman-like shock reactions in mice. J. Exp. Med. 171:1853-69 issn: 0022-1007.

31. Hill, D. B., et al., Increased plasma interleukin-6 concentrations in alcoholic hepatitis. J Lab Clin Med, 1992. 119(5): p. 547-52.

32. Hill, D. B., L. S. Marsano, and C. J. McClain, Increased plasma interleukin-8 concentrations in alcoholic hepatitis. Hepatology, 1993. 18: p. 576-580.

33. Hiramatsu, N., et al., Immunohistochemical detection of Fas antigen in liver tissue of patients with chronic hepatitis C. Hepatology, 1994. 19(6): p. 1354-9.

34. Huang, Y. S., et al., Serum levels of interleukin-8 in alcoholic liver disease: relationship with disease stage, biochemical parameters and survival. J Hepatol, 1996. 24(4): p. 377-84.

35. Iio, S., et al., Serum levels of soluble Fas antigen in chronic hepatitis C patients. J Hepatol, 1998. 29(4): p. 517-23.

36. Izaki, K. (1978) Jpn. J. Bacteriol. 33:729-742).

37. John, J. F., et al. (1986) Rev. Infect. Dis. 8:693-704).

38. Kahiwamura, S., Okamura, H. (1998), Nippon. Rinsho. 56, pp. 1798-1806.

39. Kendall, K. J. et al. (1987) J. Bacteriol. 169:4177-4183).

40. Kim S H, Eisenstein M, Reznikov L, Fantuzzi G, Novick D, Rubinstein M, Dinarello C A. Structural requirements of six naturally occurring isoforms of the IL-18 binding protein to inhibit IL-18. Proc Natl Acad Sci USA 2000;97:1190-1195.

41. Knight D M, Trinh H, Le J, Siegel S, Shealy D, McDonough M, Scallon B, Moore M A, Vilcek J, Daddona P, et al. Construction and initial characterization of a mouse-human chimeric anti-TNF antibody. Mol Immunol Nov. 30, 1993:16 1443-53

42. Kohno, K., J. Kataoka, T. Ohtsuki, Y. Suemoto, I. Okamoto, M. Usui, M. Ikeda, and M. Kurimoto. 1997. IFN-gamma-inducing factor (IGIF) is a costimulatory factor on the activation of Th1 but not Th2 cells and exerts its effect independently of IL-12. J. Immunol. 158:1541-1550.

43. Lee, M., et al., Expression of Th1 and Th2 type cytokines responding to HBsAg and HBxAg in chronic hepatitis B patients. J Korean Med Sci, 1999. 14(2): p. 175-81.

44. Lukkari, T. A., et al., Short-term ethanol exposure increases the expression of Kupffer cell CD14 receptor and lipopolysaccharide binding protein in rat liver. Alcohol Alcohol, 1999. 34(3): p. 311-9.

45. Luo, K. X., et al., In situ investigation of Fas/FasL expression in chronic hepatitis B infection and related liver diseases. J Viral Hepat, 1997. 4(5): p. 303-7.

46. Maliszewski, C. R., T. A. Sato, T. Vanden Bos, S. Waugh, S. K. Dower, J. Slack, M. P. Beckmann, and K. H. Grabstein. 1990. Cytokine receptors and B cell functions. I. Recombinant soluble receptors specifically inhibit IL-1- and IL-4-induced B cell activities in vitro. J. Immunol. 144:3028-3033.

47. Maniatis, T., in “Cell Biology: A Comprehensive Treatise, Vol. 3: Gene Expression”, Academic Press, NY, pp. 563-608 (1980).

48. Maniatis et al., Molecular Cloning: A Laboratory Manual, Cold Spring Harbor Laboratory, New York, 1982.

49. Martinez, F., et al., Ethanol and cytokine secretion. Alcohol, 1992. 9(6): p. 455-8.

50. McClain, C. J., et al., Tumor necrosis factor and alcoholic liver disease. Alcohol Clin Exp Res, 1998. 22(5 Suppl): p. 248S-252S.

51. McClain, C. J. and D. A. Cohen, Increased tumor necrosis factor production by monocytes in alcoholic hepatitis. Hepatology, 1989. 9(3): p. 349-51.

52. Micallef, M. J., T. Ohtsuki, K. Kohno, F. Tanabe, S. Ushio, M. Namba, T. Tanimoto, K. Torigoe, M. Fujii, M. Ikeda, S. Fukuda, and M. Kurimoto. 1996. Interferon-gamma-inducing factor enhances T helper 1 cytokine production by stimulated human T cells: synergism with interleukin-12 for interferon-gamma production. Eur-J-Immunol 26:1647-51 issn: 0014-2980.

53. Mizushima, S. and Nagata, S. (1990) pEF-BOS, a powerful mammalian expression vector. Nucleic Acid Res. 18:5322-5328.

54. Monteleone G, Trapasso F, Parrello T, Biancone L, Stella A, Iuliano R, Luzza F, Fusco A, Pallone F. Bioactive IL-18 expression is up-regulated in Crohn's disease. J Immunol 1999;163:143-147.

55. Muhl H, Kampfer H, Bosmann M, Frank S, Radeke H, Pfeilschifter J. Interferon-gamma mediates gene expression of IL-18 binding protein in nonleukocytic cells. Biochem Biophys Res Commun 2000;267:960-963.

56. Nakamura, K., H. Okamura, M. Wada, K. Nagata, and T. Tamura. 1989. Endotoxin-induced serum factor that stimulates gamma interferon production. Infect-Immun 57:590-5 issn: 0019-9567.

57. Nanji, A. A., et al., Activation of nuclear factor κ B and cytokine imbalance in experimental alcoholic liver disease in the rat. Hepatology, 1999. 30(4): p. 934-43.

58. Nishimura, T. and A. Ohta, A critical role for antigen-specific Th1 cells in acute liver injury in mice. J. Immunol, 1999. 162: p. 6503-6509.

59. Novick, D., B. Cohen, and M. Rubinstein. 1994. The Human Interferon alpha/beta Receptor—Characterization and Molecular Cloning. Cell 77:391-400.

60. Novick, D., B. Cohen, and M. Rubinstein. 1992. Soluble Interferon-alpha Receptor Molecules Are Present in Body Fluids. FEBS Lett 314:445-448.

61. Novick, D., H. Engelmann, D. Wallach, and M. Rubinstein. 1989. Soluble cytokine receptors are present in normal human urine. J. Exp. Med. 170:1409-1414.

62. Novick, D, Kim, S-H, Fantuzzi, G, Reznikov, L, Dinarello, C, and Rubinstein, M (1999). Immunity 10, 127-136.

63. Ohlinger, W., et al., Immunohistochemical detection of tumor necrosis factor-α, other cytokines and adhesion molecules in human livers with alcoholic hepatitis. Virchows Arch A Pathol Anat Histopathol, 1993. 423(3): p. 169-76.

64. Okamura, H., H. Tsutsui, T. Komatsu, M. Yutsudo, A. Hakura, T. Tanimoto, K. Torigoe, T. Okura, Y. Nukada, K. Hattori, K. Akita, M. Namba, F. Tanabe, K. Konishi, S. Fukuda, and M. Kurimoto. 1995. Cloning of a new cytokine that induces IFN-gamma production by T cells. Nature 378:88-91.

65. Okamoto, T., et al., Induction of Fas ligand and Fas antigen mRNA expressions in interferon-γ transgenic mouse liver. Jpn J Pharmacol, 1998. 78(2): p. 233-5.

66. Okazaki, M., et al., Hepatic Fas antigen expression before and after interferon therapy in patients with chronic hepatitis C. Dig Dis Sci, 1996. 41(12): p. 2453-8.

67. Okamoto, T., K. Yamamura, and O. Hino, The mouse interferon-γ transgene chronic hepatitis model (Review). Int J Mol Med, 1999. 3(5): p. 517-20.

68. Olee T, Hashimoto S, Quach J, Lotz M. (1999). J Immunol 162:2 1096-100

69. Parnet, P, Garka, K E, Bonnert, T P. Dower, S K, and Sims, J E. (1996), J. Biol. Chem. 271, 3967-3970.

70. Plater-Zyberk C, Bonnefoy J Y. Marked amelioration of established collagen-induced arthritis by treatment with antibodies to CD23 in vivo. Nat Med 1995;1:781-785.

71. Pizarro T T, Michie M H, Bentz M, Woraratanadharm J, Smith M F, Jr., Foley E, Moskaluk C A, Bickston S J, Cominelli F. IL-18, a novel immunoregulatory cytokine, is up-regulated in Crohn's disease: expression and localization in intestinal mucosal cells. J Immunol 1999;162:6829-6835.

72. Reimund J M, Wittersheim C, Dumont S, Muller C D, Baumann R, Poindron P, Duclos B. Mucosal inflammatory cytokine production by intestinal biopsies in patients with ulcerative colitis and Crohn's disease. J Clin Immunol 1996;16:144-150.

73. Rothe, H., N. A. Jenkins, N. G. Copeland, and H. Kolb. 1997. Active stage of autoimmune diabetes is associated with the expression of a novel cytokine, IGIF, which is located near Idd2. J-Clin-lnves.t 99:469-74 issn: 0021-9738.

74. Saha N, Moldovan F, Tardif G, Pelletier J P, Cloutier J M, Martel-Pelletier J. (1999). Arthritis Rheum 42:8 1577-87.

75. Sambrook, J., E. F. Fritsch, and M. T., Molecular Cloning: A laboratory manual. 2nd ed. ed. 1989, Cold Spring Harbor, N.Y.: Cold Spring Harbor Laboratory.

76. Simonet, W. S., et al., Osteoprotegerin: a novel secreted protein involved in the regulation of bone density. Cell, 1997. 89(2): p. 309-319.

77. Sheron, N., et al., Elevated plasma interleukin-6 and increased severity and mortality in alcoholic hepatitis. Clin Exp Immunol, 1991. 84(3): p. 449-53.

78. Sompayrac, L. H. and K. L. Danna, Efficient infection of monkey cells with DNA of simian virus 40. Proc. Nat'l. Acad. Sci. USA, 1981. 78: p. 7575-7578.

79. Sparks, C. A., et al., Assignment of the nuclear mitotic apparatus protein NuMA gene to human chromosome 11q13. Genomics, 1993. 17: p. 222-224.

80. Su, G. L., et al., CD14 and lipopolysaccharide binding protein expression in a rat model of alcoholic liver disease. Am J Pathol, 1998. 152(3): p. 841-9.

81. Taieb, J., et al., Raised plasma soluble Fas and Fas-ligand in alcoholic liver disease [letter]. Lancet, 1998. 351(9120): p. 1930-1.

82. Triantaphyllopoulos, K A, Williams, R, Tailor, H, and Chernakovsky, Y (1999). Arthritis and Rheumatism 42, 90-99.

83. Tsutsui, H., K. Nakanishi, K. Matsui, K. Higashino, H. Okamura, Y. Miyazawa, and K. Kaneda. 1996. IFN-gamma-inducing factor up-regulates Fas ligand-mediated cytotoxic activity of murine natural killer cell clones. J. Immunol. 157:3967-73 issn: 0022-1767.

84. Tsuij, H., Mukaida, N., Harada A., Kaneko, S., Matsushita, E., Nakanuma, Y., Tsutsui, H., Okamura, H., Nakanishi, K., Tagawa,m Y, Iwakura, Y., Kobayashi, K., and Matsuschima, K. (1999), J. Immunol. 162, pp. 1049-1055.

85. Tucci, A., James, H., Chicheportiche, R., Bonnefoy, J. Y., Dayer, J. M., and Zubler, R. H., 1992, J. Immunol. 148, 2778-2784.

86. Ushio, S., M. Namba, T. Okura, K. Hattori, Y. Nukada, K. Akita, F. Tanabe, K. Konishi, M. Micallef, M. Fujii, K. Torigoe, T. Tanimoto, S. Fukuda, M. Ikeda, H. Okamura, and M. Kurimoto. 1996. Cloning of the cDNA for human IFN-gamma-inducing factor, expression in Escherichia coli, and studies on the biologic activities of the protein. J. Immunol. 156:4274-4279.34. Okayama, H. and Berg, P. (1983) A cDNA cloning vector that permits expression of cDNA inserts in mammalian cells. Mol. Cell. Biol. 3:280-289.

87. Williams R O, Mason L J, Feldmann M, Maini R N. Synergy between anti-CD4 and anti-tumor necrosis factor in the amelioration of established collagen-induced arthritis. Proc Natl Acad Sci USA Mar. 29, 1994 91:7 2762-6.

88. Yasuda, H., et al., Identity of osteoclastogenesis inhibitory factor (OCIF) and osteoprotegerin (OPG): a mechanism by which OPG/OCIF inhibits osteoclastogenesis in vitro. Endocrinology, 1998. 139: p. 1329-1337.

89. Yoshimoto T, Takeda, K, Tanaka, T, Ohkusu, K, Kashiwamura, S, Okamura, H, Akira, S and Nakanishi, K (1998), J. Immunol. 161, 3400-3407.

1. Use of an IL-18 inhibitor for the manufacture of a medicament for treatment and/or prevention of liver injury.

2. The use according to claim 1, wherein the liver injury is acute.

3. The use according to claim 1, wherein the liver injury is chronic.

4. The use according to any of claim 1 to 3, wherein the liver injury is alcoholic hepatitis, viral hepatitis, immune hepatitis, fulminant hepatitis, liver cirrhosis, and primary biliary cirrhosis.

5. The use according to claim 4, wherein the liver injury is fulminant hepatitis.

6. Use of an inhibitor of IL-18 in the manufacture of a medicament for the treatment and/or prevention of arthritis.

7. The use according to claim 6, wherein the arthritis is inflammatory arthritis.

8. The use according to claim 7, wherein the inflammatory arthritis is rheumatoid arthritis.

9. Use of an inhibitor of IL-18 in the manufacture of a medicament for the treatment and/or prevention of cartilage destruction.

10. Use of an IL-18 inhibitor for the manufacture of a medicament for the treatment and/or prevention of an inflammatory bowel disease.

11. The use according to claim 10, wherein the inflammatory bowel disease is Crohn's disease.

12. The use according to claim 10, wherein the inflammatory bowel disease is ulcerative colitis.

13. The use according to any of claims 1 to 12, wherein the inhibitor of IL-18 is selected from caspase-1 (ICE) inhibitors, antibodies against IL-18, antibodies against any of the IL-18 receptor subunits, inhibitors of the IL-18 signalling pathway, antagonists of IL-18 which compete with IL-18 and block the IL-18 receptor, and IL-18 binding proteins, isoforms, muteins, fused proteins, functional derivatives, active fractions or circularly permutated derivatives thereof having essentially the same activity as an IL-18 binding protein.

14. The use according to claim 13, wherein the inhibitor of IL-18 is an IL-18 antibody.

15. The use according to claim 14, wherein the IL-18 antibody is a humanised IL-18 antibody.

16. The use according to claim 15, wherein the IL-18 antibody is a human IL-18 antibody.

17. The use according to claim 13, wherein the inhibitor of IL-18 is a IL-18 binding protein, or an isoform, a mutein, fused protein, functional derivative, active fraction or circularly permutated derivative thereof.

18. The use according to claim 17, wherein the IL-18 binding protein is PEGylated.

19. The use according to claim 17, wherein the inhibitor of IL-18 is a fused protein comprises all or part of an IL-18 binding protein fused to all or part of an immunoglobulin, and wherein the fused protein binds to IL-18.

20. The use according to claim 19, wherein the fused protein comprises all or part of the constant region of an immunoglobulin.

21. The use according to claim 20, wherein the immunoglobulin is of the IgG1 or IgG2 isotype.

22. The use according to any of the preceding claims, wherein the medicament further comprises an interferon.

23. The use according to claim 22, wherein the interferon is interferon-β.

24. The use according to claim 22 or 23, wherein the inhibitor of IL-18 is used simultaneously, sequentially, or separately with the interferon.

25. The use according to any of the preceding claims, wherein the medicament further comprises a Tumor Necrosis Factor (TNF) antagonist.

26. The use according to claim 25, wherein the TNF antagonist is TBPI and/or TBPII.

27. The use according to claim 25 or 26, wherein the inhibitor of IL-18 and/or the interferon is used simultaneously, sequentially, or separately with the TNF antagonist.

28. The use according to any of the preceding claims, wherein the medicament further comprises a COX-inhibitor.

29. The use according to claim 28, wherein the COX-inhibitor is a COX-2 inhibitor.

30. The use according to any of the preceding claims, wherein the inhibitor of IL-18 is used in an amount of about 0.0001 to 10 mg/kg of body weight, or about 0.01 to 5 mg/kg of body weight or about 0.1 to 3 mg/kg of body weight or about 1 to 2 mg/kg of body weight.

31. The use according to any of the preceding claims, wherein the inhibitor of IL-18 is used in an amount of about 0.1 to 1000 μg/kg of body weight or 1 to 100 μg/kg of body weight or about 10 to 50 μg/kg of body weight.

32. The use according to any of the preceding claims, wherein the IL-18 inhibitor is administered subcutaneously.

33. The use according to any of the preceding claims, wherein the IL-18 inhibitor is administered intramuscularly.

34. The use according to any of the preceding claims, wherein the IL-18 inhibitor is administered daily.

35. The use according to any of the preceding claims, wherein the IL-18 inhibitor is administered every other day.

36. Use of an expression vector comprising the coding sequence of an inhibitor of IL-18 in the manufacture of a medicament for the treatment and/or prevention of liver injury.

37. Use of an expression vector comprising the coding sequence of an inhibitor of IL-18 in the manufacture of a medicament for the treatment and/or prevention of arthritis.

38. Use of an expression vector comprising the coding sequence of an inhibitor of IL-18 in the manufacture of a medicament for the treatment and/or prevention of inflammatory bowel disease arthritis.

39. Use according to any of claims 36 to 38 for gene therapy.

40. Use of a vector for inducing and/or enhancing the endogenous production of an inhibitor of IL-18 in a cell in the manufacture of a medicament for the treatment and/or prevention of liver injury.

41. Use of a vector for inducing and/or enhancing the endogenous production of an inhibitor of IL-18 in a cell in the manufacture of a medicament for the treatment and/or prevention of arthritis.

42. Use of a vector for inducing and/or enhancing the endogenous production of an inhibitor of IL-18 in a cell in the manufacture of a medicament for the treatment and/or prevention of inflammatory bowel disease.

43. Use of a cell that has been genetically modified to produce an inhibitor of IL-18 in the manufacture of a medicament for the treatment and/or prevention of liver injury.

44. Use of a cell that has been genetically modified to produce an inhibitor of IL-18 in the manufacture of a medicament for the treatment and/or prevention of arthritis.

45. Use of a cell that has been genetically modified to produce an inhibitor of IL-18 in the manufacture of a medicament for the treatment and/or prevention of inflammatory bowel disease.

46. A pharmaceutical composition comprising a therapeutically effective amount of an inhibitor of IL-18 and a therapeutically effective amount of an interferon.

47. A pharmaceutical composition comprising a therapeutically effective amount of an IL-18 inhibitor and a therapeutically effective amount of a TNF antagonist.

48. A pharmaceutical composition comprising a therapeutically effective amount of an IL-18 inhibitor and a therapeutically effective amount of a COX-2 inhibitor.

49. A pharmaceutical composition comprising a therapeutically effective amount of an IL-18 inhibitor in combination with a therapeutically effective amount of any or all of an interferon, a TNF antagonist or a COX-2 inhibitor.

50. Method of treatment and/or prevention of liver injury comprising administering to a host in need thereof an effective inhibiting amount of an IL-18 inhibitor.

51. Method of treatment and/or prevention of arthritis comprising administering to a host in need thereof an effective inhibiting amount of an IL-18 inhibitor.

52. Method of treatment and/or prevention of inflammatory bowel disease comprising administering to a host in need thereof an effective inhibiting amount of an IL-18 inhibitor.
